(19) United States
(12) Patent Application Publication
(10) Pub. No.: US 2024/0010996 A1
(43) Pub. Date: Jan. 11, 2024

(54) Bacterial unspecific peroxygenases (BUPO's) and methods and uses thereof

(71) Applicants: GECCO BIOTECH B.V., Groningen (NL); ZYMVOL BIOMODELING S.L., Barcelona (ES)

(72) Inventors: Nikola Loncar, Groningen (NL); Lur Alonso Cotchico, Barcelona (ES)

(21) Appl. No.: 18/027,774

(22) PCT Filed: Sep. 23, 2021
(86) PCT No.: PCT/NL2021/050571

§ 371 Date: Mar. 22, 2023

(30) Foreign Application Priority Data
Sep. 23, 2020 (EP) 20197762.6

(51) Int. Cl. C12N 9/08; C12N 15/70; C12P 17/18

(52) U.S. Cl. CPC C12N 9/0065; C12N 15/70; C12P 17/182; C12Y 111/02001

(57) ABSTRACT

Novel polypeptides having peroxygenase activity, and methods and uses related thereto. A method for the production of melanin or a melanin-like pigment, comprising the use of a polypeptide having pigment producing activity, wherein said polypeptide is selected from the group consisting of: (a) a polypeptide comprising an amino acid sequence having at least 50% pairwise sequence identity when aligned to at least 200 consecutive amino acid residues of Seq. no. 16 and comprising at least two of the following motifs: i) RXFWXRWXXGHQ; ii) LXXLXXCXD; iii) PRXXYH; iv) RXR[ML]ALQH; v) CXXL; vi) HXXIAXH; vii) DLXHXG; and viii) VDGXHHPV; (b) a polypeptide comprising an amino acid sequence having at least 30% pairwise sequence identity when aligned to at least 150 amino acid residues of Seq. no. 12, and comprising the motif HXXXC; and c) a fragment of the polypeptide of (a) or (b) that has peroxygenase activity.

The invention relates to the field of protein engineering and biocatalysis. More in particular, it relates to novel polypeptides having peroxygenase activity, and to methods and uses related thereto.

Peroxygenases can catalyze hydroxylation and epoxidation reactions on aromatic and aliphatic carbon atoms using only hydrogen peroxide. Enzymes known so far are of fungal origin and they belong to two classes. Class I (short peroxygenases) enzymes are represented by dimeric proteins (26 kDa MroUPO/CglUPO) with histidine as charge stabilizer in the active site. They do not contain intramolecular disulfide bonds, but intermolecular disulfide bonds connect the monomers. Class II, or long, peroxygenases contain monomeric proteins of ˜44 kDa which have arginine as charge stabilizer in the active site and which contain intra-molecular disulfide bridges. Main representatives are AaeUPO and PabUPOs (Wang et al. Curr. Opinion in Chem. Biology, Vol. 37, 2017, pp. 1-9). The two classes can be identified by the presence of highly conserved motifs in their active sites (-EHD-S-E- for class I and -EGD-S-R-E for class II).

Cytochromes P450 (CYPs) are a superfamily of enzymes containing heme as a cofactor that function as monooxygenases. In mammals, these proteins oxidize steroids, fatty acids, and xenobiotics, and are important for the clearance of various compounds, as well as for hormone synthesis and breakdown. In plants, these proteins are important for the biosynthesis of defensive compounds, fatty acids, and hormones. CYP enzymes have been identified in all kingdoms of life: animals, plants, fungi, protists, bacteria, and archaea, as well as in viruses. However, they are not omnipresent; for example, they have not been found in Escherichia coli. More than 50,000 distinct CYP proteins are known.

CYPs are, in general, the terminal oxidase enzymes in electron transfer chains, broadly categorized as P450-containing systems. The term “P450” is derived from the spectrophotometric peak at the wavelength of the absorption maximum of the enzyme (450 nm) when it is in the reduced state and complexed with carbon monoxide. Most CYPs require a protein partner to deliver one or more electrons to reduce the iron (and eventually molecular oxygen).

The remarkable reactivity and substrate promiscuity of P450s have long attracted the attention of chemists. Recent progress towards realizing the potential of using P450s towards difficult oxidations have included: (i) eliminating the need for natural co-factors by replacing them with inexpensive peroxide containing molecules, (ii) exploring the compatibility of P450s with organic solvents, and (iii) the use of small, non-chiral auxiliaries to predictably direct P450 oxidation.

Fungal peroxygenases emerged as a promising group of enzymes that could replace P450s in many reactions. Better said, peroxygenases could step in instead of P450s for biocatalytic transformations and thus enable scale-up of such processes. The use of P450s is often complicated due to multi-component systems. Even in the case of BM3-like P450s, there is still dependence on cofactor NAD(P)H and significant uncoupling rate which further leads to the enzyme inactivation (Holtmann et al., ChemBioChem 2016, 17, 1391).

WO2008/119780 discloses eight different fungal peroxygenases from Agrocybe aegerita, Coprinopsis cinerea, Laccaria bicolor and Coprinus radians. Ullrich et al., 2004, Appl. Env. Microbiol. 70(8): 4575-4581 discloses a peroxygenase from the agaric basidiomycete strain Agrocybe aegerita (strain TM-A1), which was found to oxidize aryl alcohols and aldehydes. WO2006/034702 discloses methods for the enzymatic hydroxylation of non-activated hydrocarbons, such as, naphtalene, toluol and cyclohexane, using the AaP peroxygenase enzyme of Agrocybe aegerita TM A1. DE 10332065 A1 discloses methods for the enzymatic preparation of acids from alcohols through the intermediary formation of aldehydes by using the AaP peroxygenase enzyme of Agrocybe aegerita TM A1.

WO2011/120938 discloses methods for enzymatic hydroxylation in position 2 or 3 of substituted or unsubstituted, linear or branched aliphatic hydrocarbons, using various fungal peroxygenases.

Several issues remain to be solved by protein engineering of peroxygenases. These include enantio- and stereoselectivity, but also preventing or lowering the peroxidase activity. For example, once hydroxylated, aromatics give phenols which can be taken as substrates for peroxidase activity to yield phenoxy radicals as products, which radicals lead to the formation of undesired polymers. A solution proposed in the literature is to add radical scavengers and there are many examples in the literature where ascorbic acid is used for that purpose.

However, a major obstacle for improvement and (industrial) application of existing peroxygenases is the fact that they are of fungal origin. This requires yeast or fungal expression systems, which hampers the production and engineering efforts. Two distinct types of heme peroxidase enzymes from bacterial origin have been described in the art, but these are suspected to be peroxygenases. These are SfmD from Streptomyces lavendulae (Tang et al., J. Biol. Chem. Vol. 287, 2012, pp. 5112-5121) and Orf13/LmbB2 from Streptomyces refuineus (Connor et al. Biochemistry 2011, 50, 8926-8936), both involved in the synthesis of complex natural products. SfmD is involved in the synthesis of Saframycin A, and Orf13 is involved in the biosynthetic pathway of Anthramycin.

However, the enzymes involved in the synthesis of secondary metabolites were found to have a rather specific substrate usage, thus limiting their application as a broad range biocatalyst for peroxygenase-type reactions.

The inventors therefore set out to identify novel enzymes of non-fungal origin and possessing a high substrate promiscuity, i.e. displaying peroxygenase activity against a diverse set of (commercially relevant) substrates. Ideally, the enzyme can be expressed at a high level in a non-fungal host cell. Furthermore, they aimed at providing enzymes and methods for production of melanin-type pigments using recombinant microbial host cells, e.g. to increase the natural melanogenic capacity of an organism or to generate novel melanin-producing (bacterial) strains.

At least some of these goals were met by the identification of a set of novel bacterial unspecified peroxygenases (BUPOs) that were found to be applicable in various reactions of interest for industrial use. One subfamily of novel BUPOs showed some sequence similarity to the Orf13/LmbB2, while a second subfamily showed some sequence similarity to SfmD. Notably however, the novel BUPOs were found to have distinct catalytic properties as compared to the known enzymes. These encompass the hydroxylation or oxidation of a substituted or unsubstituted, linear or branched, aliphatic or aromatic substrate. In particular, the inventors identified BUPOs having the capacity to produce dark, melanin-type, pigments when expressed in bacterial host cells. Other useful novel applications of the BUPOs include the enantioselective sulfoxidation of an optionally substituted alkyl sulfide, aryl sulfide or aryl alkyl sulfide substrate, the manufacture of a substituted or unsubstituted indigo dye, and the oxidation of a primary alcohol.

Accordingly, in one aspect the invention relates to a (biotechnological) method for the production of melanin and/or melanin-like pigment(s), comprising the use of a polypeptide selected from the group consisting of:
(a) a polypeptide comprising an amino acid sequence having at least 50% pairwise sequence identity when aligned to at least 200, preferably at least 250, more preferably at least 270 consecutive amino acid residues of Seq. no. 16 of FIG. 1, and comprising at least two of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV;
wherein X is any amino acid;
(b) a polypeptide comprising an amino acid sequence having at least 30% pairwise sequence identity when aligned to at least 150, preferably 180, more preferably 220 consecutive amino acid residues of Seq. no. 12 of FIG. 2, and comprising the motif HXXXC, wherein X is any amino acid, preferably H[IRKAQVG][GNELSYRHM][VI]C, more preferably HARVC;
(c) a fragment of the polypeptide of (a) or (b) that has pigment producing activity.

The term “melanin or a melanin-like pigment” is meant to encompass a groups of polymeric brown black colored pigments that are widely found in nature, and which are the products of the enzyme-catalyzed oxidation of phenolic or indolic substrates. In particular, they include eumelanin, pheomelanin, allomelanins and pyomelanins.

The term “pigment producing activity” as used herein refers to the capacity to catalyse the formation of one or more melanin or a melanin-like pigments from its precursor(s).

In a specific aspect, the method comprises hydroxylation of L-tyrosine to L-DOPA and subsequent oxidation to dopachrome and the formation of melanin or a melanin-like pigment. Alternative or additional substrate(s) include L-cysteine and N-(hydroxyphenyl)glycine.

The invention also relates to a method for the hydroxylation or oxidation of a substituted or unsubstituted, linear or branched, aliphatic or aromatic substrate, comprising contacting the substrate with a polypeptide having peroxygenase activity and a source of hydrogen peroxide, wherein said polypeptide is selected from the polypeptides under (a) and (b) as defined herein above, or a fragment of the polypeptide of (a) or (b) that has the desired peroxygenase activity.

The term “having peroxygenase activity” as used herein refers to the capacity to catalyse the following reaction S+H₂O₂→SO+H₂O, S being the substrate to be hydroxylated. For example, if S is depicted as RH, the reaction to be catalyzed is RH+H₂O₂→ROH+H₂O.

Preferably, the method comprises one or more of the following:
the enantioselective sulfoxidation of an optionally substituted alkyl sulfide, aryl sulfide or aryl alkyl sulfide substrate;
the manufacture of a substituted or unsubstituted indigo dye by contacting a substituted or unsubstituted indole substrate with a source of hydrogen peroxide and the polypeptide;
oxidation of a primary alcohol.

The term “pairwise sequence identity percentage” generally means the coefficient between amino acid residue positions that have the same amino acid in two aligned sequences over all positions when the two protein sequences are aligned. Percent (%) sequence identity with respect to amino acid sequences disclosed herein is defined as the percentage of amino acid residues in a candidate sequence that are pair-wise identical with the amino acid residues in a reference sequence, i.e. a protein molecule or fragment of the present disclosure, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using public available computer software such as pairwise sequence identity when aligned using the Global alignment with free end gaps method, BLAST, ALIGN, or Megalign (DNASTAR) software. Those skilled in the art can determine appropriate parameters for measuring alignment, including any algorithms needed to achieve maximum alignment over the full length of the sequences being compared.

The term “amino acid” or “amino acid residue” refers to an α- or β-amino carboxylic acid. When used in connection with a protein or peptide, the term “amino acid” or “amino acid residue” typically refers to an α-amino carboxylic acid having its art recognized definition such as an amino acid selected from the group consisting of: L-alanine (Ala or A); L-arginine (Arg or R); L-asparagine (Asn or N); L-aspartic acid (Asp or D); L-cysteine (Cys or C); L-glutamine (Gln or Q); L-glutamic acid (Glu or E); glycine (Gly or G); L-histidine (His or H); L-isoleucine (ILE or I): L-leucine (Leu or L); L-lysine (Lys or K); L-methionine (Met or M); L-phenylalanine (Phe or F); L-proline (Pro or P); L-serine (Ser or S); L-threonine (Thr or T); L-tryptophan (Trp or W); L-tyrosine (Tyr or Y); and L-valine (Val or V), although modified, synthetic, or rare amino acids such as e.g. taurine, ornithine, selenocysteine, homocystine, hydroxyproline, thioproline, iodotyrosine, 3-nitro-tyrosine, ornithine, citrulline, canavanine, 5-hydroxytryptophane, carnosine, cycloleucine, 3,4-dihydroxy phenylalanine, N-acetylcysteine, prolino 1, allylglycine or acetidine-2-carboxylic acid may be used as desired. Generally, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr).

A “fragment” as used herein refers to a portion of a parental protein which portion has peroxygenase activity. Such a fragment can comprise consecutive amino acids of the parental protein. A “fragment” can also refer to a protein in which fragments of a parental protein are fused together. A fragment can also comprise modifications such as amino acid substitutions, amino acid deletions or amino acid insertions compared to the parental protein.

A method of the invention is not disclosed or suggested in the art.

U.S. Pat. No. 7,291,490 relates to bacterial enzymes involved in the biosynthesis of benzodiazepines. One of the enzymes disclosed (UNKV or ORF13; referred to as SEQ ID NO:26) showing some sequence similarity to representative BUPO of Seq. no. 16 of the present invention. ORF13 is demonstrated to have tyrosine 3-hydroxylase activity and is used to convert tyrosine to L-DOPA. WO02/101051 also discloses this enzyme and suggests it has the ability to hydroxylate L-tyrosine to L-3,4 dihydroxyphenylalanine in a first step of a cascade of reactions involving several other enzymes, resulting in the formation of lincomycins or anthramycins. However, nothing is taught or suggested to use the ORF13 enzyme in the production of melanin-type pigments, or any of the other hydroxylation/oxidation reactions of the present invention.

Martinez et al. (Frontiers in Bioeng. and Biotech. 2019, Vol. 7, Art. 285) provide a summary and discussion of approaches made toward the generation of recombinant melanin-producing micro-organisms and production processes related thereto. Reference is made to melanogenic enzymes mainly being tyrosinases and laccases, both being copper-containing enzyme. However, some tyrosinases including the tyrosinase from Streptomyces sp. require chaperons for copper insertion. The enzyme 4-hydroxyphenylacetic acid (4-HPA) hydroxylase is a two-component flavin adenine dinucleotide (FAD)-dependent monooxygenase which can hydroxylate various monohydric and dihydric phenols and therefore it can show melanogenic activity. Importantly, the cited art is silent about heme-containing melanin forming enzymes as disclosed in the present invention. Even more surprisingly, the melanogenic activity observed in Streptomyces is attributed in the art only to tyrosinase (Lin et al., J Microbiol Immunol Infect. 2005 October; 38(5):320-6; Guo et al., FEMS Microbiol Lett. 2015 April; 362(8)), whereas the present inventors identified the Streptococcus enzyme of Seq. no. 15 as one of the most potent pigment producing enzymes. Thus, while tyrosinases and laccases can be considered a natural tool of microbes for melanin production, the present inventors are the first to demonstrate that some of the tightly regulated enzymes from secondary metabolism pathways like tyrosine-hydroxylases, or herein renamed to BUPOs, can facilitate efficient melanin production when expressed in a heterologous hosts.

UniProt entry A0A3N4UNC0 discloses an amino acid sequence obtained from genomic DNA of P. pacifica. The sequence is referred to as a hypothetical protein. Whereas it has 100% sequence identity with representative BUPO of Seq. no. 12 of the present invention, the disclosure fails to teach or suggest that the protein can actually be produced and has a technical function. Therefore, the art is silent on the use of the protein in hydroxylation or oxidation methods as demonstrated in the present invention.

In one embodiment, a method of the invention involves the use of an “LmbB2-type” or “Type II” polypeptide comprising an amino acid sequence having at least 50% pairwise sequence identity when aligned to at least 200 consecutive amino acid residues of Seq. no. 16 (see Table 1), and comprising at least two of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV;

wherein X is any amino acid.

Preferably, at least motifs i) and vi) are present.

Type II enzymes were found to have a highly active and versatile peroxygenase activity. See FIG. 1 for an alignment of exemplary Type II enzymes of the invention, including the motifs that appear to be conserved.

Exemplary polypeptides of the invention comprise at least three, preferably four, more preferably five, even more preferably at least six of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV.

For example, provided is a polypeptide comprising the motifs RXFWXRWXXGHQ, preferably R[LV]FWYRWIAGHQ; LXXLXXCXD, preferably L[DE][ALV]L[ACST][TAS]C[IV]D; PRXXYH, preferably PR[AD][HQ]YH; RXR[ML]ALQH, preferably R[APT]R[ML]ALQH; CXXL, preferably C[EAR][AE]L; HXXIAXH, preferably H[DS][HF]IA[ND]H; DLXHXG, preferably DL[AS]H[NH]G; and VDGXHHPV, preferably VDG[AR]HHPV.

In addition, the polypeptide under (a) may furthermore comprises one or both of the following motifs:
a. LWRAM
b. EDL[YF]DN[FY][FY], preferably EDLYDNFF.

A Type II enzyme for use in the present invention may comprise or consist of an amino acid sequence having at least 50%, 55%, 60%, 65%, 70%, 75%, 80%, 85%, 90%, 92%, 95%, 96%, 97%, 98% or 99% pairwise sequence with any one of the sequences shown in Table 1 (Seq. no. 16, 15, 17 or 18; Group II enzymes), or a fragment thereof that has peroxygenase activity. In one aspect, a Type II enzyme comprises or consists of an amino acid sequence having at least 50%, 55%, 60%, 65%, 70%, 75%, 80%, 85%, 90%, 92%, 95%, 96%, 97%, 98% or 99% pairwise sequence identity when aligned to at least 200, preferably at least 250, more preferably at least 280 consecutive amino acid residues of Seq. no. 16.

In one aspect, the enzyme contains one or more of the residues corresponding to Arg115, Met118, Phe125, Ser 126, Leu180, Tyr200 and Phe204 of Seq. no. 16, since these residues were found to be important for recombinant expression and/or catalytic activity, in particular for melanin production. For example, the enzyme contains one or more of the residues corresponding to Phe125, Ser 126, Leu180, Tyr200 and Phe204 of Seq. no. 16. In one specific aspect, at least Tyr200 is present, preferably in combination with one or more of Arg115, Met118, Phe125, Ser 126, Leu180 and Phe204. In another embodiment, at least Arg115 and/or Leu180 are present, or similar residues such as Lys115, Val180 or Ile180.

The Trp residues at positions 50, 53 and/or 110 of Seq. no. 16, may be replaced with another “bulky” residue, such as Phe, Tyr or Arg.

In one embodiment, the polypeptide is an enzyme according to any one of Seq.no. 15-18, preferably Seq. no. 16, or a fragment thereof that has the desired peroxygenase/pigment producing activity.

In another embodiment, the invention involves an “SfmD-type” or “Type I” polypeptide comprising an amino acid sequence having at least 30% pairwise sequence identity when aligned to at least 150 amino acid residues of Seq. no. 12, preferably starting from residue Leucine 156 of Seq. no. 12, and comprising the motif HXXXC, wherein X is any amino acid. See FIG. 2 for an alignment of exemplary Type I enzymes of the invention.

The HXXXC motif is of relevance for heme incorporation in the enzyme. In one embodiment, the Type I enzyme comprises the motif H[IRKAQVG][GNELSYRHM][VI]C, preferably HARVC.

The Type I polypeptide preferably furthermore comprises one or more of the following residues/motifs:
i) DXXFXXXR; preferably D[LEAFDSRHT][AGFHY]F[GNCLR][IAV][VELKIRSD]R, more preferably DSYFLVER;
ii) R[WR]XX[GQ]HXXF; preferably R[WR][VIRMKH][RYCLVQK][GQ]-H[HLYQR][VLIAS]F, more preferably RWKQQHQLF;
iii) YXXXXR[PV]; preferably Y[N/T/Q/V/E/A/H/D/R/Q][E/T/D/S/Q/A]-[Q/R/E/S/A/I/G/F/T/V/M/L/N][IV]RP, more preferably YESRIRP;
iv) H232;
v) [LM]281; preferably L281;
wherein X is any amino acid and wherein the numbering corresponds to the amino acid sequence of Seq. no. 12.

In one embodiment, the polypeptide comprises the motif HXXXC, wherein X is any amino acid, and at least motif i) as defined above. For example, it comprises at least motifs i), ii) and iii), or motifs i) and ii), or motifs i) and iii). Exemplary enzymes include those with Seq. no. 2-13, and 19-22 (see Table 2).

In one embodiment, the polypeptide comprises the motif HXXXC, wherein X is any amino acid, and at least motif(s) ii), iii), iv) and/or v), for example at least motifs ii) and iii), or motifs iii) and iv), or motifs ii), iv) and v). In a preferred embodiment, at least motif HXXXC and H232 (motif iv) are present. In a specific aspect, the polypeptide does not contain motif i). Exemplary enzymes include those with Seq. no. 23-29.

In one embodiment, the Type I polypeptide comprises the residues/motifs:
i) DXXFXXXR; preferably D[LEAFDSRHT][AGFHY]F[GNCLR][IAV][VELKIRSD]R, more preferably DSYFLVER;
ii) R[WR]XX[GQ]HXXF; preferably R[WR][VIRMKH][RYCLVQK][GQ]-H[HLYQR][VLIAS]F, more preferably RWKQQHQLF;
iii) YXXXXR[PV]; preferably Y[N/T/Q/V/E/A/H/D/R/Q][E/T/D/S/Q/A]-[Q/R/E/S/A/I/G/F/T/V/M/L/N][IV]RP, more preferably YESRIRP;
iv) H232; and
v) [LM]281.

A Type I polypeptide may comprise or consist of an amino acid sequence having at least 35%, 40%, 45%, 50%, 55%, 60%, 65%, 70%, 75%, 80%, 85%, 90%, 92%, 95%, 96%, 97%, 98% or 99% pairwise sequence identity with any one of Seq. no. 2-13 and 19-29 (Group I enzymes), or a fragment thereof that has peroxygenase activity. Preferably, the polypeptide shows at least 75%, 80%, 85%, 90%, 92%, 95%, 97%, 98% or 99% pairwise sequence identity with any one of Seq. no. 2-7, 10-13, 19-21, and 23-29, preferably with Seq. no. 12.

Exemplary enzymes include polypeptides having a sequence according to any one of Seq. no. 2-13 and 19-29, preferably Seq. no. 12, or a fragment thereof that has peroxygenase activity. Preferred Type I enzymes include those comprising or consisting of any one of sequences 2, 3, 4, 7, 8, 11, 12, 13, 20, 21 and 28, or a fragment thereof that has peroxygenase activity.

A polypeptide according to the invention may comprise (by genetic fusion) one or more additional amino acid sequences or protein tag(s) at its N- and/or C-terminus. In one embodiment, the polypeptide comprises an N-terminal tag. In another embodiment, the polypeptide comprises a C-terminal tag. In a further embodiment, the polypeptide comprises both an N- and a C-terminal tag. The additional tag sequence(s) may aid in the expression yield, folding, solubilization, purification and/or immobilization of the polypeptide. Such sequences are well known in the art. Exemplary fusion tags include maltose binding protein, N-utilization substance A (NusA), glutathione S-transferase (GST), biotin carboxyl carrier protein, thioredoxin, and cellulose binding domain, short peptide tags such as oligohistidine (6×His; His-tag), oligolysine, S-peptide, and the FLAG peptide. Exemplary solubility tag includes SUMO (Small Ubiquitin-like Modifier) or MBP (maltose-binding protein). In a specific aspect, the enzyme contains an N-terminal His-tag. Alternatively, or additionally, it is provided with a SUMO tag.

The tag sequence(s) may be (proteolytically) removed from the polypeptide prior to their application to catalyze a peroxygenase reaction. For example, SUMO fusion proteins can be cleaved to remove the SUMO moiety using SUMO-specific proteases such as Ulp1.

The invention also relates to a composition comprising one or more polypeptide(s) according to the invention. For example, the composition comprises whole cells, permeabilized cells, a cell extract or a cell-free extract comprising a recombinantly expressed enzyme of the invention. In a preferred aspect, the composition is a bacterial cell culture comprising a bacterial host cell expressing one or more polypeptides of the invention as heterologous enzyme. In another embodiment, the composition comprises the enzyme(s) in a soluble or immobilized form. The composition may be a reaction mixture comprising one or more peroxygenases, one or more substrates, a source of H₂O₂, and/or products.

Also disclosed is an isolated polynucleotide encoding a polypeptide according to the invention. The polynucleotide may be comprised in a nucleic acid construct or expression vector, preferably wherein the polynucleotide is operably linked to one or more control sequence(s) that direct the production of the polypeptide in an expression host. Exemplary expression vectors are known in the art. The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like. In one embodiment, the vector is an E. coli expression vector. For example, polypeptides can be expressed using a pET-based (IPTG-inducible) vector or a pBAD-based (arabinose inducible) vector.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The control sequence may also be a leader, a non-translated region of an mRNA that is important for translation by the host cell. The leader is operably linked to the 5′-terminus of the polynucleotide encoding the polypeptide. Any leader that is functional in the host cell may be used. The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention. Preferred terminators for bacterial host cells are obtained from the genes for Bacillus clausii alkaline protease {aprH), Bacillus licheniformis alpha-amylase (amyL), and Escherichia coli ribosomal RNA (rrnB). The control sequence may also be an mRNA stabilizer region downstream of a promoter and upstream of the coding sequence of a gene which increases expression of the gene. Examples of suitable mRNA stabilizer regions are obtained from a Bacillus thuringiensis crylllA gene and a Bacillus subtilis SP82 gene.

A further embodiment of the invention relates to a recombinant host cell comprising the nucleic acid construct or expression vector of the invention encoding a polypeptide as herein disclosed. In one aspect, the encoding nucleic acid sequence is part of an expression vector. In another embodiment, the encoding nucleic acid sequence is integrated in the genome of the host cell. For example, it is possible to integrate the encoding gene into the genome of a host organism by methods known in the art, including genome editing methods, homologous recombination, and methods involving the CRISPR Cas system.

The host cell may be any cell useful in the recombinant production of a polypeptide of the present invention, e.g. a prokaryote or a eukaryote. The prokaryotic host cell may be any Gram-positive or Gram-negative bacterium. Gram-positive bacteria include Bacillus, Clostridium, Enterococcus, Geobacillus, Lactobacillus, Lactococcus, Oceanobacillus, Staphylococcus, Streptococcus, and Streptomyces. Gram-negative bacteria include Campylobacter, E. coli, Flavobacterium, Fusobacterium, Helicobacter, Ilyobacter, Neisseria, Pseudomonas, Salmonella, and Ureaplasma.

In a specific aspect, the host cell is E. coli. For expression with pET-based vectors E. coli BL21, E. coli C41/C43 or E. coli BL21AI strains can be used, while for pBAD-based vectors E. coli NEB10beta, E. coli TOP10, E. coli, BL21AI and other standard strains be used.

Host cells may be genetically modified to have characteristics that improve genetic manipulation, protein secretion, protein stability and/or other properties desirable for expression or secretion of a peroxygenase enzyme. For example, host cells may be modified to contain an enzyme capable of removing a tag sequence that is fused to a polypeptide of the invention. For example, the host cell comprises a vector that encodes not only a SUMO- and His-tagged peroxygenase of interest, but also SUMO-tagged Ulp1 protease. Co-expression of these two proteins results in the in vivo cleavage of the enzyme of interest from the SUMO tag, while still leaving the enzyme of interest in a form that can be purified from a soluble cell lysate by nickel affinity chromatography.

Also provided is a method of producing a polypeptide having peroxygenase activity, comprising (a) cultivating said host cell under conditions conducive for production of the polypeptide; and (b) recovering the polypeptide. Suitable media for growing the host of the invention are well known in the art, for example, see Sambrook et al., Molecular Cloning (1989), supra. In general, a suitable media contains all the essential nutrients for the growth of the host system. The media can be supplemented with antibiotics that are selected for host-vector system.

An expressed polypeptide can be used in the form of whole cells, permeabilized cells, a cell extract or a cell-free extract comprising an enzyme of the invention. In another embodiment, the enzyme is used in a soluble or immobilized form. Expressed enzyme(s) may be recovered from cells using methods known in the art. Optionally, a protein can be enriched for (e.g., purified or partially purified) using methods well known in the art. For example, the polypeptide may be isolated by conventional procedures including centrifugation, filtration, extraction, spray-drying, evaporation, chromatography (e.g., ion exchange, solid phase binding, affinity, hydrophobic interaction, chromatofocusing, and size exclusion chromatography) and/or filtration, or precipitation. Protein refolding steps can be used, as desired, in completing the configuration of the mature protein. Finally, high performance liquid chromatography (HPLC) can be employed in the final purification steps.

The invention also provides a method for hydroxylation or oxidation of a substrate of interest, comprising contacting the substrate with a source of hydrogen peroxide and a polypeptide according to the invention having peroxygenase activity. Preferably, the substrate is a hydrocarbon substrate, more preferably a substituted or unsubstituted, linear or branched, aliphatic or aromatic substrate.

The hydrogen peroxide required by the peroxygenase may be provided as an aqueous solution of hydrogen peroxide or a hydrogen peroxide precursor for in situ production of hydrogen peroxide. Any solid entity which liberates upon dissolution a peroxide, which is useable by peroxygenase, can serve as a source of hydrogen peroxide. Compounds which yield hydrogen peroxide upon dissolution in water or an appropriate aqueous based medium include metal peroxides, percarbonates, persulphates, perphosphates, peroxyacids, alkyperoxides, acylperoxides, peroxyesters, urea peroxide, perborates and peroxycarboxylic acids or salts thereof.

Another source of hydrogen peroxide is a hydrogen peroxide generating enzyme system, such as an oxidase together with a substrate for the oxidase. Examples of combinations of oxidase and substrate comprise, but are not limited to, amino acid oxidase (see e.g. U.S. Pat. No. 6,248,575) and a suitable amino acid, glucose oxidase (see e.g. WO95/29996) and glucose, lactate oxidase and lactate, galactose oxidase (see e.g. WO00/50606) and galactose, formate oxidase and formate (Willot et al.; 2020, ChemCatChem Volume12, Issue10, pp. 2713-2716) and aldose oxidase (see e.g. WO99/31990) and a suitable aldose.

Hydrogen peroxide or a source of hydrogen peroxide may be added at the beginning of or during a method of the invention, e.g. as one or more separate additions of hydrogen peroxide; or continuously as fed-batch addition. Typical amounts of hydrogen peroxide correspond to levels of from 0.001 mM to 25 mM, preferably to levels of from 0.005 mM to 5 mM, and particularly to levels of from 0.01 to 1 mM or 0.02 to 2 mM hydrogen peroxide. Hydrogen peroxide may also be used in an amount corresponding to levels of from 0.1 mM to 25 mM, preferably to levels of from 0.5 mM to 15 mM, more preferably to levels of from 1 mM to 10 mM, and most preferably to levels of from 2 mM to 8 mM hydrogen peroxide. The method of the invention may be carried out with an immobilized peroxygenase.

Herewith, the invention also relates to the use of a polypeptide according to the invention as a catalyst, preferably as a catalyst of a peroxygenase or melanin-type pigment producing reaction.

A method of the invention may be carried out in an aqueous solvent or buffered system (reaction medium). Suitable buffered systems are easily recognized by one skilled in the art, and include K-phosphate (K-Pi) buffers.

The methods according to the invention may be carried out at a temperature between 0 and 90° C., preferably between 5 and 80° C., more preferably between 10 and 70° C., even more preferably between 15 and 60° C., most preferably between 20 and 50° C., and in particular between 20 and 40° C. The methods of the invention may employ a treatment time of from 10 seconds to (at least) 24 hours, preferably from 1 minute to (at least) 12 hours, more preferably from 5 minutes to (at least) 6 hours, most preferably from 5 minutes to (at least) 3 hours, and in particular, from 5 minutes to (at least) 1 hour.

In one embodiment, the invention provides a method for hydroxylation of a substrate of interest. For example, a peroxygenase as herein disclosed is suitably used to catalyze the hydroxylation of a substituted phenol or phenolic acid. In a specific aspect, an enzyme of Seq.no. 7, 15, 16, 17 or 18, or an active fragment thereof, is used.

It was surprisingly found that peroxygenases of the invention are suitably used in the synthesis of L-DOPA and Melanin. L-DOPA, also known as levodopa or l-3,4-dihydroxyphenylalanine, is an amino acid that is made and used as part of the normal biology of humans, as well as some animals and plants. Humans, as well as a portion of the other animals that utilize L-DOPA in their biology, make it via biosynthesis from the amino acid L-tyrosine. L-DOPA is the precursor to the neurotransmitters dopamine, norepinephrine (noradrenaline), and epinephrine (adrenaline), which are collectively known as catecholamines. Furthermore, L-DOPA itself mediates neurotrophic factor release by the brain and CNS. L-DOPA in its pure form is sold as a psychoactive drug under the non-proprietary name “levodopa”. As a drug, it is used in the clinical treatment of Parkinson's disease and dopamine-responsive dystonia.

Accordingly, in a specific aspect, the invention provides a method for the hydroxylation of L-tyrosine to L-DOPA, optionally further comprising oxidation to dopachrome and the formation of melanin. Of particular interest for this reaction are polypeptides comprising or consisting of a sequence that has at least 40%, at least 50%, at least 60%, or at least 70% pairwise sequence identity with any one of Seq. no. 2, 8, 11, 12, 13, 16, 17 or 20, preferably 12 or 16, or a fragment thereof that has the desired L-Tyr hydroxylation activity. Herewith, the invention provides a method for the biotechnological production of L-DOPA.

Melanin plays an important role in protecting human body from the harmful effects of ultraviolet rays. Melanin is also an important factor in medical science and cosmetology. It is known that melanin is formed or synthesized in skin tissues. Excessive amounts of melanin darken the skin, and the nonuniform distribution of melanin causes chloasma and ephelis, both of which are skin disorders. The biosynthesis pathway of melanin involves the catalytic hydroxylation of tyrosine to L-3,4-dihydroxyphenylalanine (L-DOPA) and the conversion of L-DOPA to dopachrome.

In a specific aspect, the invention provides a method for the production of melanin or a melanin-like pigment. Of particular interest for this reaction are polypeptides comprising or consisting of a sequence that has at least 40%, at least 50%, at least 60%, or at least 70% pairwise sequence identity with any one of Seq. no. 2, 8, 11, 12, 13, 15, 16, 17, 18 and 20, preferably Seq. no. 15, 16 or 17, or a fragment thereof that has the desired pigment producing activity. Herewith, the invention provides a method for the biotechnological production of melanin and/or melanin-related brown or black pigment(s).

With the application of genetic engineering techniques, it is possible to overexpress one or more genes encoding a BUPO in accordance with the present invention that is involved in melanin formation in a microbial host cell. This allows for an improvement in the productivity of melanogenic organisms, as well as allowing the generation of novel recombinant microbial strains that can produce diverse types of melanins. Furthermore, the metabolic engineering of microbial hosts by modifying pathways related to the supply of melanogenic precursors can provide strains with the capacity of performing the total synthesis of melanins from simple carbon sources in the scale of grams.

In one embodiment, the invention provides a method for the fermentative production of melanin or a melanin-like pigment, comprising the steps of:
i) providing a microbial host cell expressing the BUPO having pigment producing activity as heterologous polypeptide;
ii) culturing the host cell in a culture medium and allowing for the production of melanin or melanin-like pigment; and
iii) isolating melanin or melanin-like pigment.

For example, polypeptides can be expressed as heterologous enzyme a pET-based vector or a pBAD-based vector. In one embodiment, a pET-based vector is used.

Preferred BUPOs for use in the fermentative production of melanin include those having at least 60%, or at least 70%, or at least 80%, preferably at least pairwise sequence identity of Seq. no. 2, 8, 11, 12, 13, 15, 16, 17, 18 and 20, preferably Seq. no. 15, 16 or 17, or a fragment thereof that has the desired pigment producing activity. For example, it was found that the residues corresponding to Leu180 and Arg115 are important for melanin production. In a specific aspect, it is the polypeptide of Seq. no. 16 or a variant thereof showing pigment producing activity, for instance a mutant wherein Trp50 and/or Trp53 is changed into a “bulky” residue such as Arg or Phe.

The host cell can be a bacterial, yeast or fungal host cell, preferably a bacterial host cell, more preferably E. coli. Processes for obtaining melanin with these organisms may involve experimental optimization methods aimed at identifying culture conditions and media components that positively impact the productivity. For example, culture parameters, such as temperature, pH, oxygen, and melanin precursor concentrations have been found to contribute to productivity. A positive correlation with pigment production can be observed by increasing in culture media the concentration of L-tyrosine or components that contain it. Thus, a method of the invention may comprise using a culture medium that has been supplemented with at least one melanin precursor, preferably L-Tyrosine. However, in most cases, typical culture media includes yeast extract or protein hydrolysates such as Tryptone. Therefore, during the melanin formation process, some media components in addition to L-tyrosine can be incorporated into polymeric pigments, yielding a pigment that is not pure eumelanin.

Thus, host cells can be grown in defined media with glucose or glycerol as carbon source to produce melanin that is of higher purity and/or easier to purify. Furthermore, such media can be supplemented with melanin precursor(s) such as tyrosine to produce eumelanin or with a mix of precursors, e.g. tyrosine, cysteine and/or N-hydroxyphenyl)glycine to produce melanin with different characteristics.

There are various ways known in the art for isolating or purifying a melanin-type pigment from a microbial cell culture, e.g. using low pH precipitation or chromatographic methods.

Accordingly, in a further embodiment the invention provides the use of a bacterial enzyme for the production of melanin or a melanin-like pigment, wherein said enzyme is a polypeptide selected from the group consisting of:
(a) a polypeptide comprising an amino acid sequence having at least 50% pairwise sequence identity when aligned to at least 200 consecutive amino acid residues of Seq. no. 16 of FIG. 1, and comprising at least two of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV;
wherein X is any amino acid;
(b) a polypeptide comprising an amino acid sequence having at least 30% pairwise sequence identity when aligned to at least 150 consecutive amino acid residues of Seq. no. 12 of FIG. 2, and comprising the motif HXXXC, wherein X is any amino acid, preferably H[IRKAQVG][GNELSYRHM][VI]C, more preferably HARVC;
(c) a fragment of the polypeptide of (a) or (b) that has pigment producing activity.

In particular, it provides the use of the enzyme wherein said bacterial enzyme is comprised in whole cells, preferably recombinant microbial cells expressing the enzyme as heterologous enzyme.

In another aspect, the invention relates to the oxidation of a primary alcohol, preferably the oxidation of veratryl alcohol to veratryl aldehyde. Of particular interest for this reaction are polypeptides comprising or consisting of a sequence that has at least 40%, at least 50%, at least 60%, or at least 70% pairwise sequence identity with any one of Seq. no. 12, 15, 16, 17 or 18, or a fragment thereof that has the desired alcohol oxidation activity.

In yet another aspect, the invention relates to polypeptides catalyzing sulfoxidation. Provided is a method for the enantioselective sulfoxidation of an optionally substituted alkyl sulfide, aryl sulfide or aryl alkyl sulfide substrate, comprising contacting the substrate with a source of hydrogen peroxide and a polypeptide according to the invention having the desired sulfoxidation activity. For example, the substrate is selected from the group consisting of methyl phenyl sulfide, benzyl phenyl sulfide, allyl phenyl sulfide, benzyl methyl sulfide, N-butyl methyl sulfide, ethyl phenyl sulphide and isopropyl phenyl sulphide. Enzymes of particular interest for sulfoxidation include those showing at least 40%, at least 50%, at least 60%, or at least 70% pairwise sequence identity with any one of Seq. no. 4, 15, 16, 17 or 18, or a fragment thereof that has the desired (thioanisole) sulfoxidation activity.

The invention also provides a method for preparing a substituted or unsubstituted indigo dye, comprising contacting a substituted or unsubstituted indole with a source of hydrogen peroxide and a polypeptide according to the invention. Of particular interest for this reaction are polypeptides comprising or consisting of a sequence that has at least 40%, at least 50%, at least 60%, or at least 70% pairwise sequence identity with any Type II enzyme as herein disclosed, in particular any one of Seq. no. 15, 16, 17 or 18, or a fragment thereof that has the desired indole hydroxylation activity.

Further aspects of the invention:
1. An isolated polypeptide having peroxygenase activity, selected from the group consisting of:
   (a) a polypeptide comprising an amino acid sequence having at least 50% pairwise sequence identity when aligned to at least 200 consecutive amino acid residues of Seq. no. 16 of FIG. 1, and comprising at least two of the following motifs:
   i) RXFWXRWXXGHQ, preferably R[LV]FWYRWIAGHQ;
   ii) LXXLXXCXD, preferably L[DE][ALV]L[ACST][TAS]C[IV]D;
   iii) PRXXYH, preferably PR[AD][HQ]YH;
   iv) RXR[ML]ALQH, preferably R[APT]R[ML]ALQH;
   v) CXXL, preferably C[EAR][AE]L;
   vi) HXXIAXH, preferably H[DS][HF]IA[ND]H;
   vii) DLXHXG, preferably DL[AS]H[NH]G; and
   viii) VDGXHHPV, preferably VDG[AR]HHPV;
wherein X is any amino acid;
   (b) a polypeptide comprising an amino acid sequence having at least 30% pairwise sequence identity when aligned to at least 150 consecutive amino acid residues of Seq. no. 12 of FIG. 2, and comprising the motif HXXXC; preferably H[IRKAQVG][GNELSYRHM][VI]C, more preferably HARVC, wherein X is any amino acid;
   (c) a fragment of the polypeptide of (a) or (b) that has peroxygenase activity.
2. The polypeptide of aspect 1 (b), furthermore comprising one or more of the following residues/motifs:
i) DXXFXXXR; preferably D[LEAFDSRHT][AGFHY]F[GNCLR][IAV][VELKIRSD]R, more preferably DSYFLVER;
ii) R[WR]XX[GQ]HXXF; preferably R[WR][VIRMKH][RYCLVQK][GQ]-H[HLYQR][VLIAS]F, more preferably RWKQQHQLF;
iii) YXXXXR[PV]; preferably Y[N/T/Q/V/E/A/H/D/R/Q][E/T/D/S/Q/A]-[Q/R/E/S/A/I/G/F/T/V/M/L/N][IV]RP, more preferably YESRIRP;
iv) H232;
v) [LM]281; preferably L281
wherein X is any amino acid and wherein the numbering corresponds to the amino acid sequence of Seq. no. 12.
3. The polypeptide of aspect 1, comprising a sequence that has at least 60%, at least 70%, at least 80%, or at least 90% pairwise sequence identity with any one of Seq. no. 15, 16, 17 and 18 of Table 1 (Group II enzymes), or a fragment thereof that has peroxygenase activity.
4. The polypeptide of aspect 3, wherein the sequence is any one of Seq. no. 15, 16, 17 and 18, preferably Seq. no. 16, or a fragment thereof that has peroxygenase activity.
5. The polypeptide of aspect 1 or 2, comprising a sequence that has at least 40%, at least 50%, at least 60%, or at least 70% pairwise sequence identity with any one of Seq. no. 2-13 and 19-29 of Table 2, or a fragment thereof that has peroxygenase activity.
6. The polypeptide of aspect 5, wherein the sequence is any one of Seq. no. 2, 3, 4, 7, 8, 11, 12, 13, 20, 21 and 28, or a fragment thereof that has peroxygenase activity.
7. The polypeptide according to any one of aspects 1-6, further comprising an N- and/or C-terminal protein tag allowing for enhanced expression, solubilization, purification and/or immobilization.

### LEGEND TO THE FIGURES

FIG. 1: Amino acid sequence alignment of a number of novel Type II BUPO polypeptides. Conserved sequence motifs (i) through (viii), and (a) and (b) are indicated on top.

FIG. 2: Amino acid sequence alignment of a number of novel Type I BUPO polypeptides. Conserved sequence motifs (i) through (v) and HXXC are indicated on top.

FIG. 3: Phylogenetic tree showing the evolutionary relationships among novel BUPOs of the invention. Known enzymes SfmD (Seq. no. 1) and lmbB2 (Seq. no. 14) are also included.

FIG. 4: UV-Vis spectra of exemplary purified BUPOs. Presence of the Soret band at ˜405 nm shows heme incorporation. Panel A: Seq. no. 12; panel B: Seq. no. 7; panel C: Seq. no. 11; panel D: Seq. no. 18; panel E: Seq. no. 17; panel F: Seq. no. 16.

FIG. 5: HPLC chromatogram of L-tyrosine conversion to L-DOPA by enzyme having Seq.no.12, Seq.no.17 or Seq.no.16. Standard for L-tyrosine has a retention time of 2.50 min, while L-DOPA standard (*) elutes at retention time of 2.41 min. Most of the L-tyrosine was converted to L-DOPA by Seq. no. 12 and 16, while for Seq. no. 17 a partial conversion was observed.

FIG. 6: Screening of novel BUPO enzymes for peroxygenase activity against different substrates. Rows A, B: melanin production from tyrosine; rows C, D: Indigo production from indole. Rows E, F: veratryl alcohol oxidation; rows G, H: p-cresol oxidation. In all cases, the reaction mixture contained 5 mM substrate in 50 mM K-phosphate buffer pH 7.5 and 1-20 μM enzyme solution. Reactions were started by addition of hydrogen peroxide (2 mM final concentration). Picture was taken after 1h. Enzymes tested: Seq.1 (SfmD)-A1, C1, E1, G1; Seq.2—A2, C2, E2, G2; Seq.5—A3, C3, E3, G3; Seq.3—A4, C4. E4, G4; Seq.13—A5, C5, E5, G5; Seq.4—A6, C6, E6, G6; Seq.12—A7, C7, E7, G7; Seq.7—B1, D1, F1, H1; Seq.11—B2, D2, F2, H2; Seq.15—B3, D3, F3, H3; Seq.18—B4, D4, F4, H4; Seq.17—B5, D5, F5, H5; Seq.16—B6, D6, F6, H6; Control (no enzyme)—B7, D7, F7, H7. Colored products were observed in wells A6, A7, B5 and B6 corresponding to melanin production; D3, D4, D5 and D6 correspond to indigo production; H5 and H6 correspond to p-cresol oxidation and polymerization.

FIG. 7: Representative HPLC chromatograms of reaction mixtures obtained by reacting exemplary BUPO enzymes with various substrates.
Panel (A) reaction of p-cresol with enzyme Seq.no.7, Seq.no.16 or Seq.no.17. p-Cresol standard has a retention time of 9.38 min, while observed hydroxylation/oxidation products (*) eluted at retention times of 2.85 min (for no. 7) and at 8.05 min and 8.55 min (for no. 16 and no. 17). Enzyme having Seq. no. 16 converted most of the substrate under given conditions.
Panel (B) reaction of m-cresol with enzyme Seq.no. 16 or Seq.no. 17. Standard for m-cresol has a retention time of 9.40 min, while observed products (*) eluted at retention times 7.6 min, 8.05 min and 8.55 min.
Panel (C) reaction of p-nitrophenol with enzymes Seq.no. 16 or Seq.no. 17. Standard for p-nitrophenol has a retention time of 3.5 min, while observed products (*) eluted at retention times 1.25 min, 5.4 min and 5.75 min.
Panel (D) reaction of protocatechuic acid with enzyme Seq.no. 15, Seq.no. 16, Seq.no. 17 or Seq.no. 18. Standard for protocatechuic acid has a retention time of 1.2 min, while observed products (*) eluted at retention times 5.1 min, 5.4 min and 7.0 min.

FIG. 8: Representative GCMS chromatograms of reaction mixtures obtained by reacting methyl phenyl sulfide (thioanisole) with exemplary BUPO enzymes. Thioanisole has a retention time of 12.42 min, while the observed oxidation product methyl phenyl sulfoxide eluted at retention time 17.50 min. Panel A: control (no enzyme); panel B: Seq. no. 4; panel C: Seq. no. 15; panel D: Seq. no. 16; panel E: Seq. no. 17; panel F: Seq. no. 18.

FIG. 9: Supernatants of the cultures of microbial cells expressing representative pigment-producing BUPOs cultured in defined media supplemented with tyrosine.

FIG. 10: Effect of mutations in polypeptide of Seq. no. 16 on the melanin producing activity. Dark color correlates with the level of melanin-type pigment.

### EXPERIMENTAL SECTION

### Example 1: Cloning, Expression and Purification of Novel BUPOs

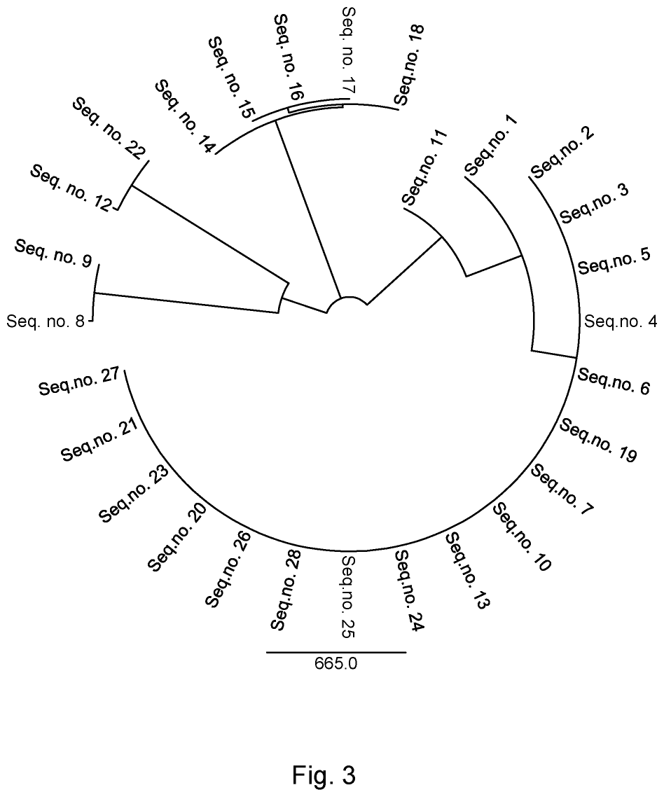

Using either SfmD or LmbB2 as query sequences, an extensive BLAST search was performed against entire NCBI database of non-redundant sequences. Then, a phylogenetic tree (FIG. 3) was constructed for SfmD-like (Type I) and Orf13/LmbB2-like (Type II) enzymes. Representatives of distinct branches were selected and ordered as synthetic genes.

Synthetic genes coding for the selected putative BUPOs were cloned either in pET28a, pBAD-His or pBAD-His-SUMO vectors. Following the confirmation of the sequence by Sanger sequencing, the final constructs were transformed into chemically competent E. coli BL21 strains (C43 or BL21AI) for pET28-based constructs or into a E. coli NEB10B for pBAD-based constructs. Single colonies were picked and grown overnight in 5 mL Luria Bertani (LB) media supplemented with corresponding antibiotic.

On the following day, the over-night culture was diluted 1:100 in terrific broth (TB) with corresponding antibiotic. Cultures were incubated at 37° C. until OD600=0.6. Expression was induced at this point by addition of 1 mM IPTG (for pET28 constructs) or 0.02 w/v % arabinose (for pBAD-constructs), together with 0.5 mM 5-aminolevulinic acid (a heme precursor); all concentrations are given as final concentrations. Expression was carried out using baffled Erlenmeyer flasks at 17° C. for 46-72 hours in orbital shakers at 150-200 rpm with 2.5-5 cm orbital.

Cells were harvested using a cooling centrifuge at 4000×g at 4° C. Cell pellets were resuspended in K-phosphate (KPi) buffer 50 mM pH 7.8 with 150 mM NaCl and with addition of 0.1 mM PMSF and 0.1 mg/ml lysozyme. Cells were disrupted using VibraCell sonicator (5 sec on, 10 sec off, 5 min total time, 70% amplitude). Clarified cell-free extract (CFE) was obtained by using cooling centrifuge at 19000×g at 4° C.

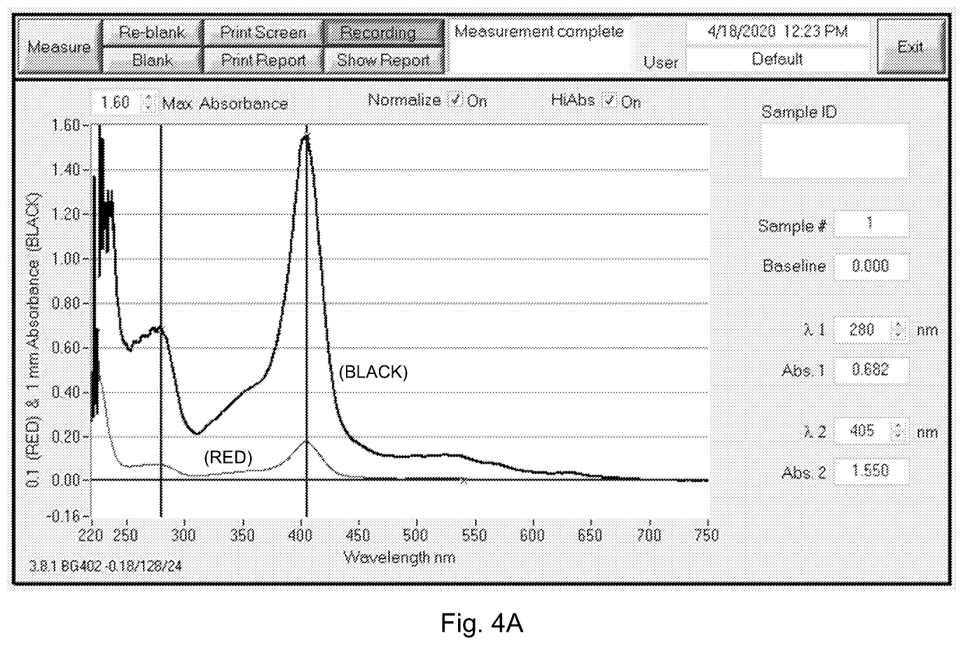

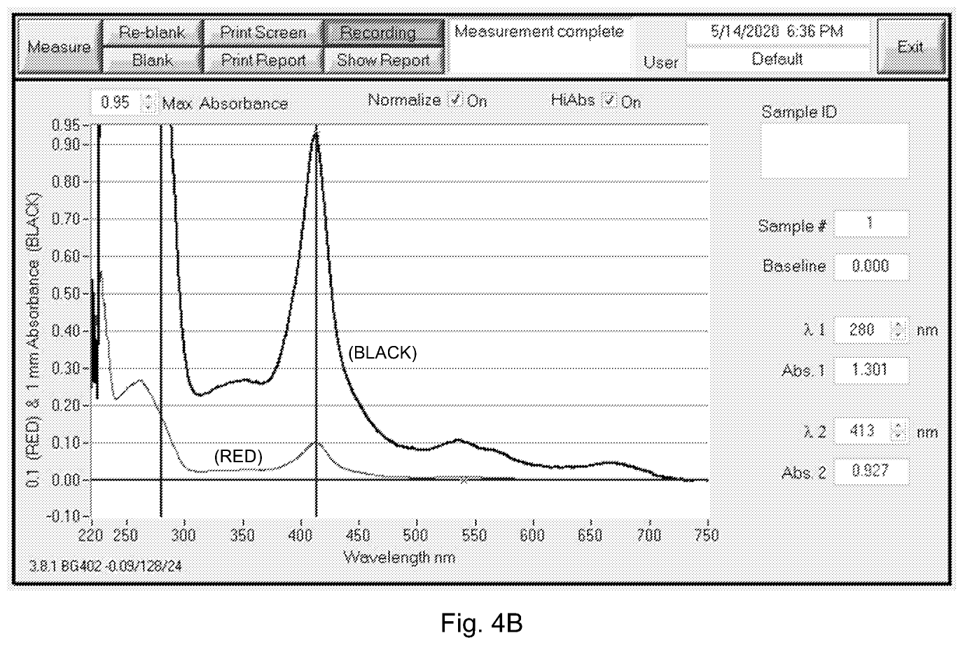

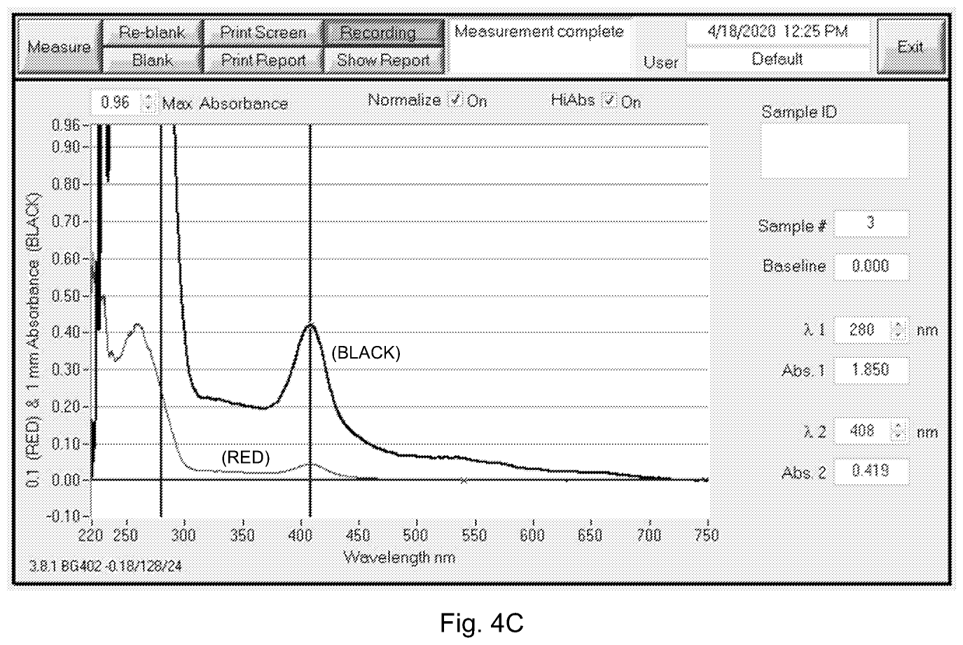

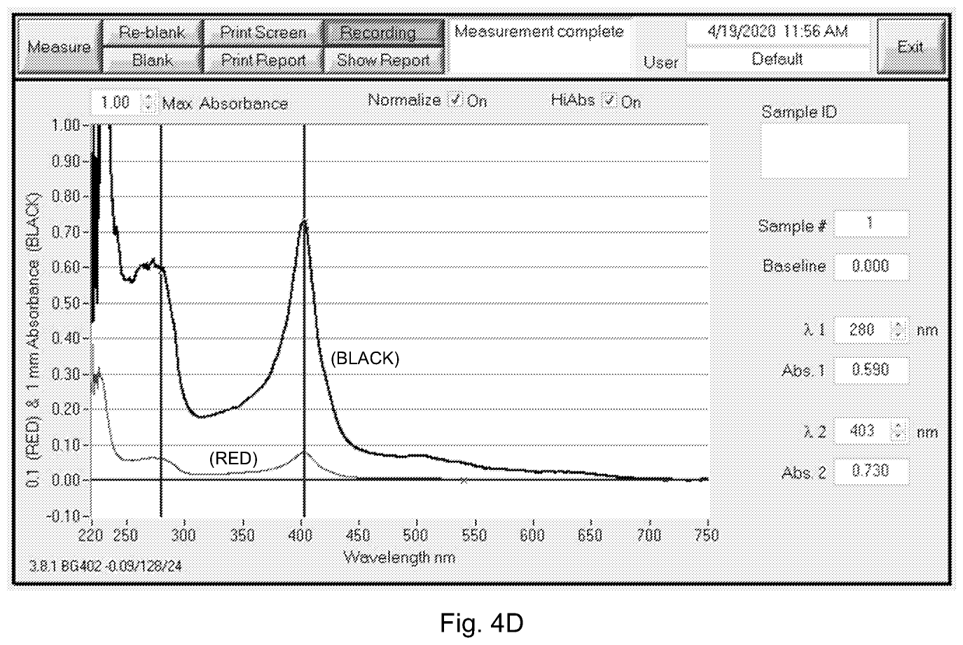

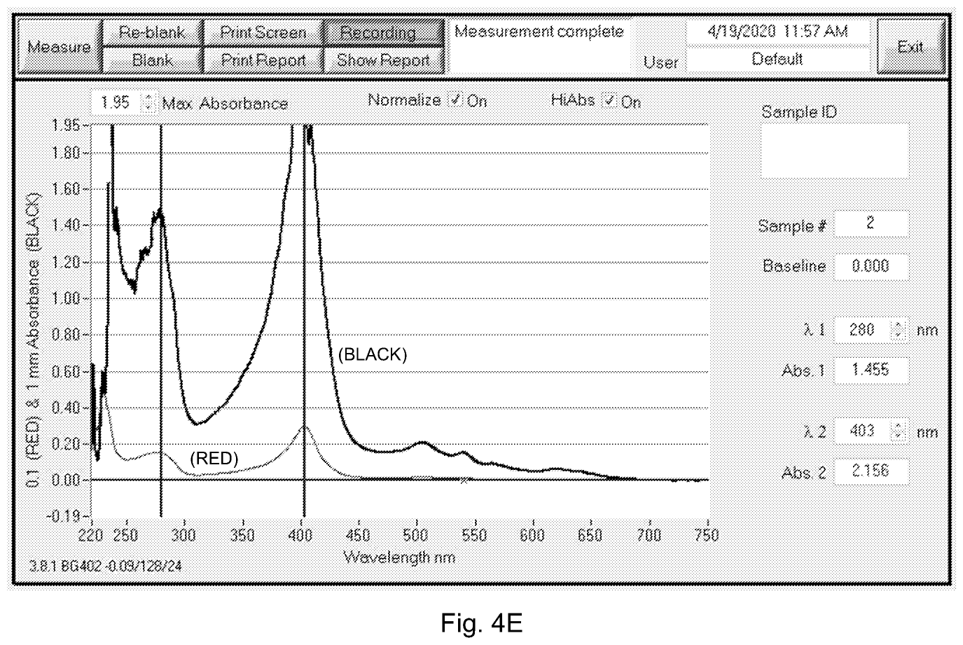

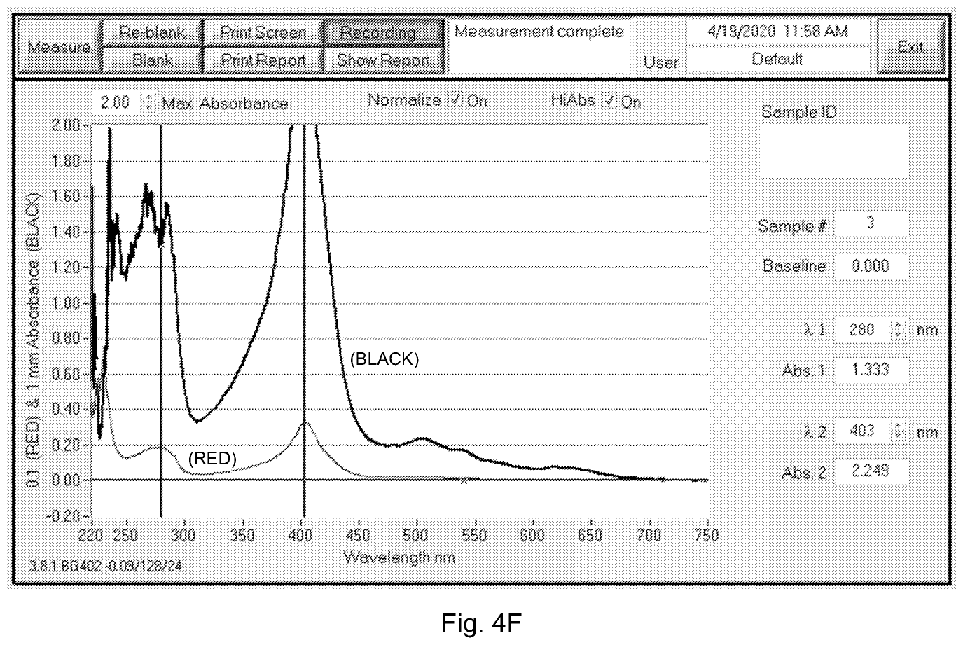

CFE was loaded on a preequilibrated Ni-Sepharose column (KPi buffer 50 mM pH 7.8 with 150 mM NaCl). Unbound proteins were washed with 3 CV of starting buffer, followed by 3 CV of starting buffer containing 30 mM imidazole, and eluted in starting buffer containing 0.5 M imidazole. Eluted fractions were pooled and buffer was exchanged using the EconoPac desalting columns (BioRad), into a KPi buffer 50 mM pH 7.8; 150 mM NaCl. UV-Vis spectra were collected to estimate the concentration of the purified protein. Proteins were then flash-frozen in liquid nitrogen and stored at −70° C. It was possible to purify most of BUPOs with a yield of ˜60 mg/L of terrific broth (TB) media. These enzymes were red and show the presence of Soret band in their UV-Vise spectra which is a proof of heme incorporation. See FIG. 4 for the spectra of some exemplary enzymes.

### Example 2: Qualitative Analysis of Peroxygenase Activity

The first batch of purified enzymes was tested for peroxygenase activity using the established Russig's blue assay (Yamada et al. (2017) PLoS ONE 12(4):e0175846). The standard assay mixture comprised 15% (v/v) ethanol, 100 mM KPi (pH 7.5), an excess (˜5-10 mM) of H₂O₂, 1-methoxynaphthalene (1-MN), and 10 μL purified enzyme in a total volume of 100 μl. The reaction was started by the addition of the enzyme and was carried out for 5 min at room temperature. The production of the reaction product Russig's blue was determined from the increase in the absorbance at 610 nm [(c) 1.45×104 M−1 cm−1]. The assay was performed in a plate reader.

It was found that at least the Type II enzymes of Seq. no. 15, 17 and 18 can utilize 1-MN as a substrate, while the SfmD enzyme known in the art did not accept 1-MN as a substrate. It was furthermore interesting to note that, whereas enzymes SphingoUPO nor SpinosaUPO (Seq. no. 11 and 13) were not active against 1-MN, their expression in bacterial host cells did give rise to a dark/black coloured culture medium. This points to the different substrate scopes between the selected enzymes.

### Example 3: Hydroxylation of L-Tyr to L-DOPA and Melanin

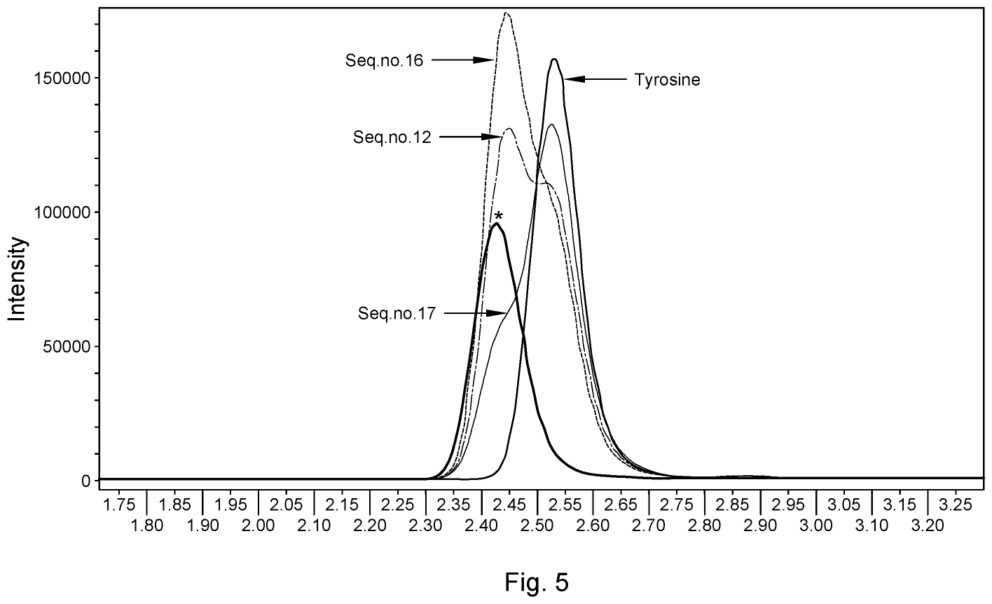

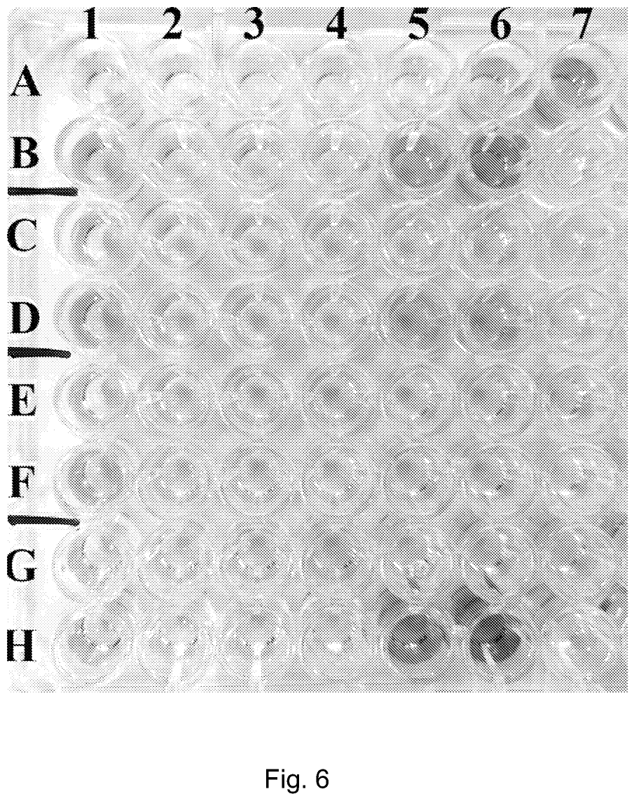

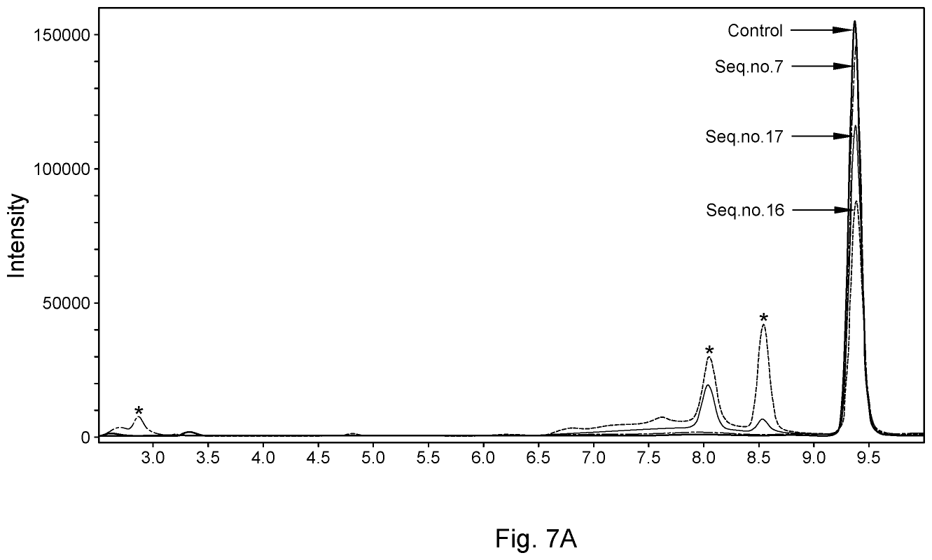

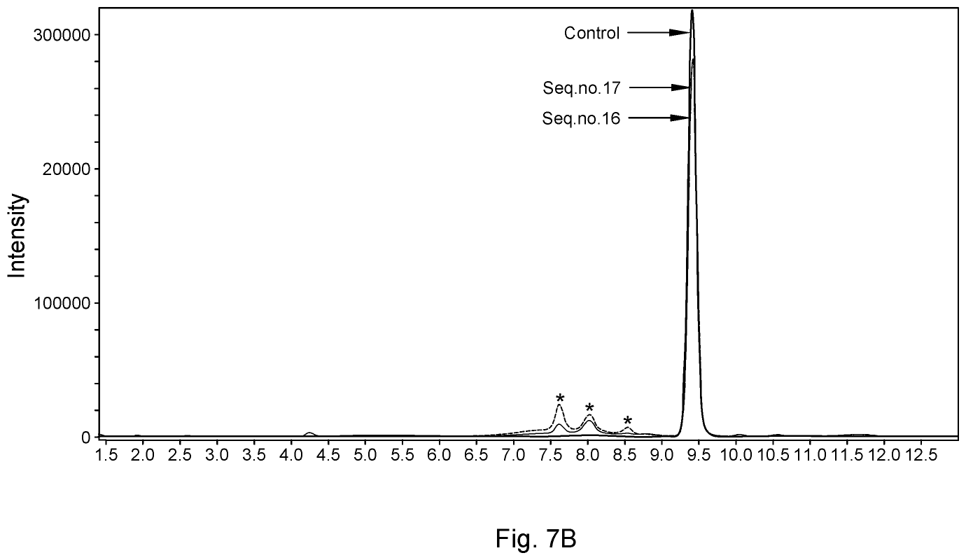

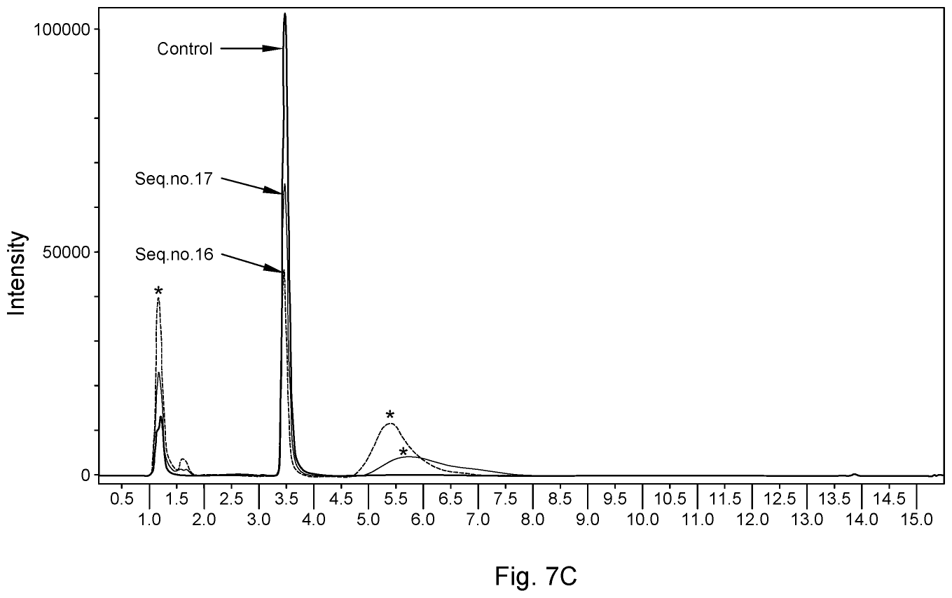

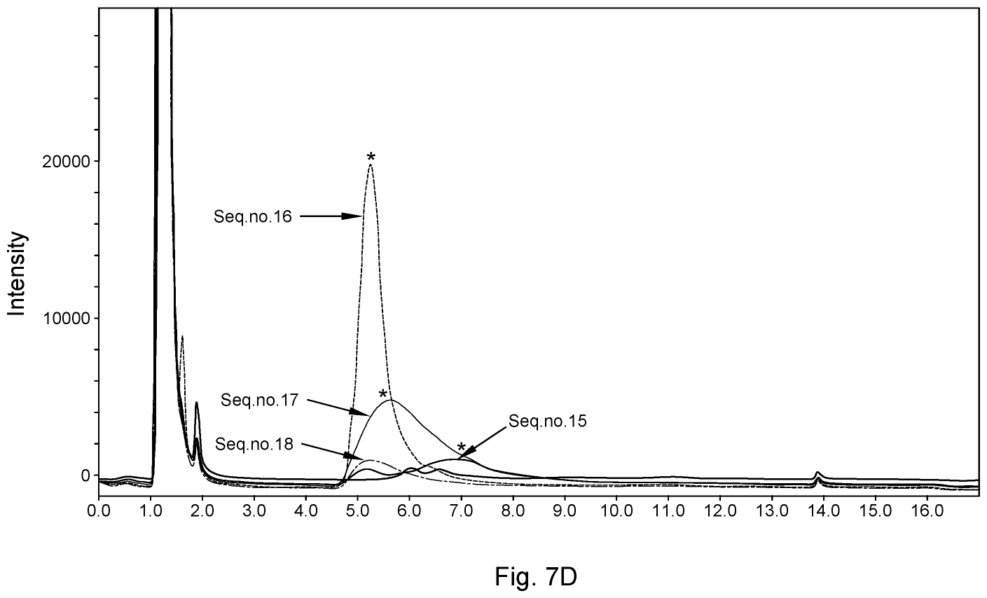

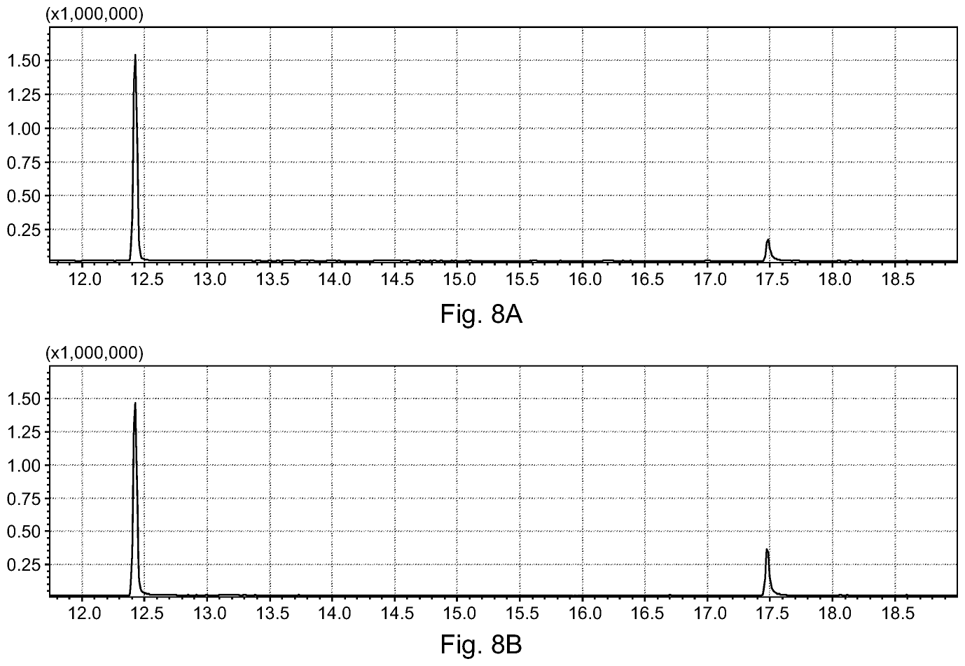

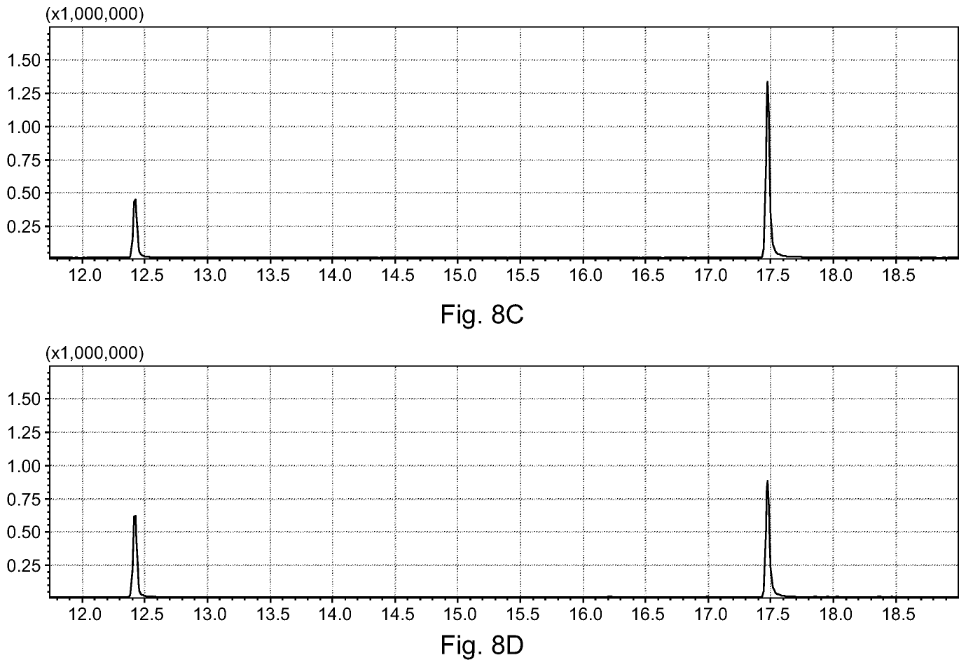

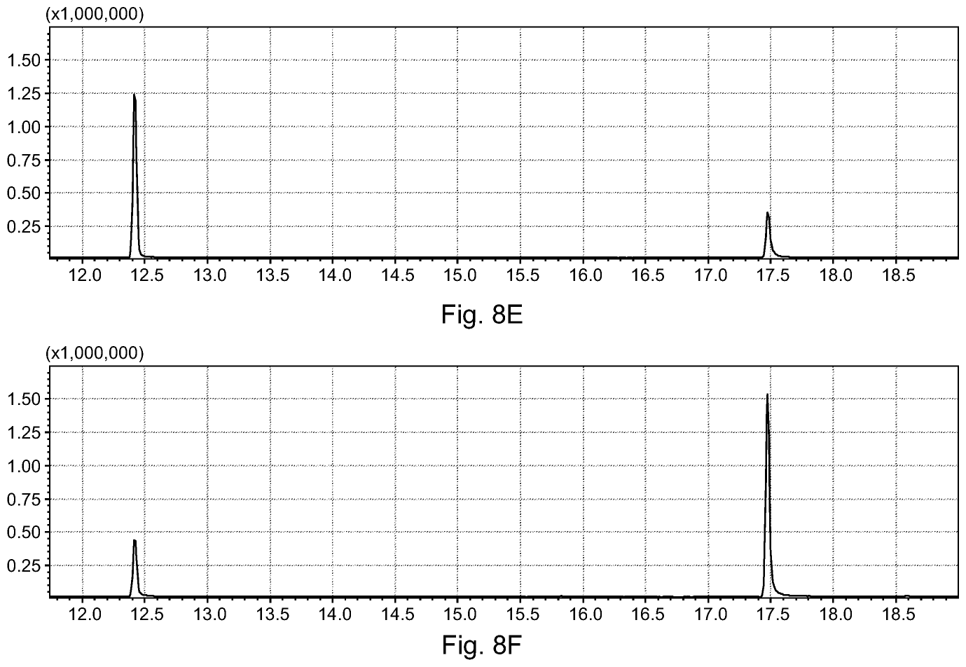

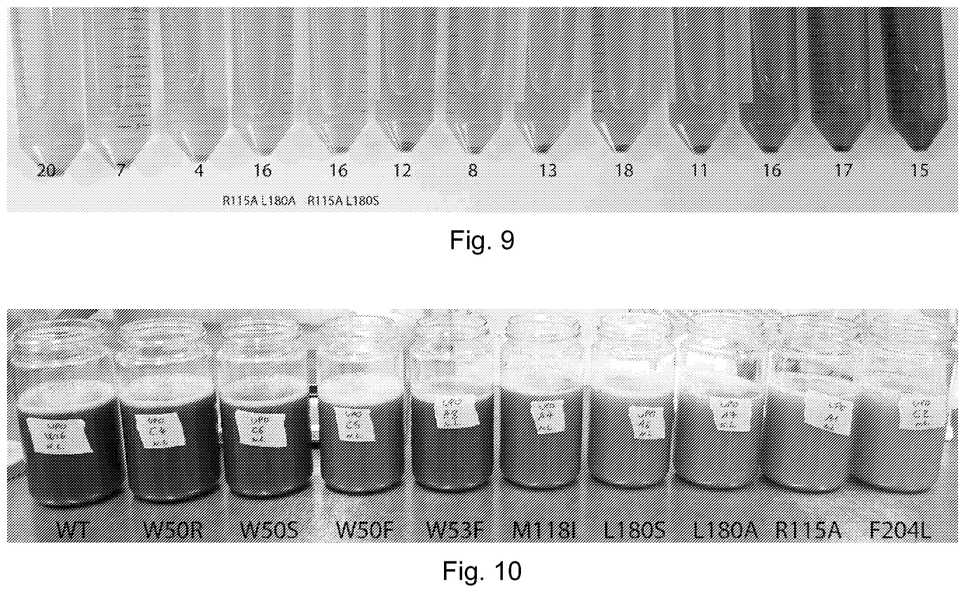

Reaction mixtures (0.5 mL) consisting of 5 mM L-Tyr in 50 mM KPi pH 7.5, 5-20 μM of the purified enzyme (seqs 12, 16 and 17) and 1 mM H₂O₂ were incubated for 1 h at 25° C. and stopped by heating at 95° C. for 5 min. Reaction mixtures were centrifuged for 5 min at 15000×g and the resulting supernatant was analyzed by reverse-phase HPLC-DAD using Waters Xselect CSH Fluoro-phenyl 5 μm 4.6×250 mm column with a linear gradient (10-50%) of water with 0.8% formic acid (A) and acetonitrile (B) over 18 min with a flow rate of 1.2 mL/min. L-Tyr and commercially available L-DOPA were used as standards. All enzymes tested were found to give L-DOPA (see FIG. 5) and melanin (see FIG. 6, rows A, B) as products, which means that these enzymes possess monophenolase and diphenolase activity.

Some of the enzyme-expressing bacterial cultures were colored dark, indicating the ability of the expressed proteins to produce dark (brown/black) pigment, melanin-like, that either can diffuse through the cell membrane or it is formed in the media from the reaction products which can diffuse through the cell. The building block is most likely tyrosine taken from the metabolic pathways.

Appearance of a dark color was not observed for enzymes known in the art (SfmD, Seq.no.1 and LmbB2, Seq.no.14), but clearly for various representatives of the newly discovered enzymes (Seq. numbers 2, 8, 11, 13, 16, 17 and 20). The observed pigment could pass through an ultrafiltration membrane with a molecular weight cut-off (MWCO) of 30 kDa. Experiments were carried out with individual purified enzymes to test whether similar result can be achieved in vitro (FIG. 6).

Reaction scheme for conversion of tyrosine to L-DOPA and dopachrome:

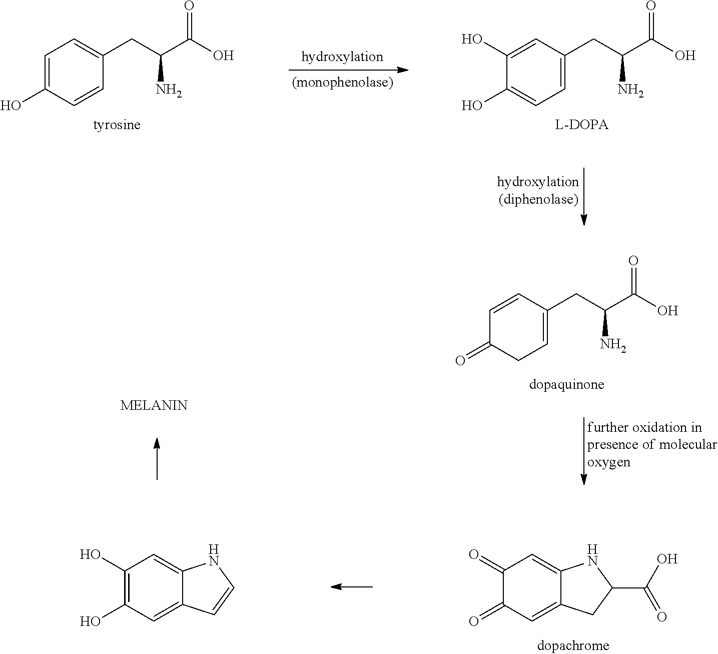 [SMILES: N[C@@H](Cc1ccc(O)cc1)C(=O)O ; N[C@@H](Cc1ccc(O)c(O)c1)C(=O)O ; N[C@@H](CC1=CCC(=O)C=C1)C(=O)O ; Oc1cc2cc[nH]c2cc1O ; O=C1C=C2CC(C(=O)O)NC2=CC1=O]

### Example 4: Hydroxylation of Substituted Phenols and Phenolic Acids

Using the same procedure as described above, hydroxylation and further oxidation to oligomers and insoluble polymers was shown for p-cresol (FIG. 6, rows G,H; and FIG. 7A) and m-cresol (FIG. 7B) with enzymes 12, 15, 16, 17 and 18, using reverse-phase HPLC. Insoluble polymers were removed by centrifugation at 15000×g for 5 min prior to HPLC analysis. Hydroxylation of p-nitrophenol (FIG. 7C) and protocatechuic acid (FIG. 7D) was confirmed by HPLC as well. For p-nitrophenol, activity was observed using enzymes with sequence numbers 12, 16 and 17, while for protocatechuic acid the activity was confirmed for enzymes with sequence numbers 12, 15, 16, 17 and 18.

### Example 5: Fermentative Production of Melanin-Type Pigment(s)

Methods

Production of melanin was tested using E. coli cells overexpressing different BUPOs in either pET or pBAD-based vectors. For pET28-based constructs E. coli BL21 cells were used in autoinduction media supplemented with kanamycin and 5-aminolevulinic acid. For the pBAD-based constructs E. coli NEB10 beta cells were used in TB media supplemented with ampicillin, 5-aminolevulininc acid and 0.02% arabinose. Expression was done by mixing 100 μL of pre-inoculum (overnight culture) with 900 μL of abovementioned media and expression was carried out in deep-well microtiter plates (DWMTP) for 20h at 30° C. Furthermore, the same experiment was carried out with addition of L-tyrosine (1 g/L) to the media. Autoinduction TB composition (g/L): Tryptone (12), Yeast Extract (24), (NH₄)₂SO₄ (3.3), MgSO₄ (0.15), Glucose (0.5), Lactose (2.0) with addition of K-phosphate buffer (14.85, same composition as standard TB-buffer).

Results

In the first set of experiments (without addition of tyrosine to the media), dark melanin-type pigment formation was observed only in the culture using pET28 constructs. The pigment formation was observed in cultures of host cells expressing enzymes corresponding to Seq. no.: 7, 8, 12 or 20 (lower intensity), or Seq. no. 11, 18, 17, 16 and 15 (strong intensity).

When the medium was supplemented with tyrosine, some of the cultures with pBAD-based constructs also showed the melanin production. These were cultures corresponding to the following seq.no.: 13, 12 and 2 (strong intensity). The cultures of host cells expressing BUPOs from a pET construct gave same result with increase in the melanin production for all the cultures, most notably for cultures of enzymes 12, 8 and 20.

Following the experiment of fermentative melanin production in complex media (TB and autoinduction media), the melanin production was evaluated in minimal media (M9+trace elements, a recipe known in the art) with glucose as a sole carbon source and supplemented with 0.5 g/L tyrosine. The expression was performed using E. coli BL21 cells expressing the heterologous BUPO polypeptides from pET28 constructs. Induction was performed using 1 mM IPTG with addition of 0.5 mM 5-aminolevulinic acid. The enzymes tested in this experiment included the following: Seq. no. 20, 7, 4, 16 (two double mutants: R115A/L180A and R115A/L180S), 12, 8, 13, 18, 11, 16, 17 and 15.

Resulting cultures showed a range of melanin production, which could be visually observed based on the colored pigment (darker cultures correspond to higher amounts of melanin). Unexpectedly, some of the cultures were able to produce larger amounts of melanin than others. For example, as is shown in FIG. 9, the cultures of enzymes with Seq. no. 20, 7 or 4, and two mutants of enzyme 16 produced melanin only relatively small amounts; cultures of enzymes 12, 8, 13, 18 and 11 produced moderate amounts of melanin, while cultures of enzymes 16, 17 and 15 produced melanin in large amounts.

### Example 6: Mutagenesis Study

Next, the polypeptide of Seq.no. 16 was used as representative pigment-producing enzyme in a mutagenesis study designed to identify residue(s) that are relevant for the manufacture of melanin-type pigments.

Changes to alanine and to other similar residues or residues present in the phylogenetic study were assessed. These included Trp50, Trp53, Tyr110, Arg115, Met118, Phe125, Ser126, Leu180, Tyr200 and Phe204. Some mutations were found to significantly affect the expression level of the polypeptide. For example, no protein could be produced from constructs encoding one of the following single point mutants: Tyr110Ala, Trp53Ala, Phe125Ala, Tyr200His, Tyr200Leu, Tyr200Val, Tyr200Ala, Phe204His and Phe204Ala. The remaining mutants could be expressed and purified, although for some the expression yield was reduced (data not shown).

Furthermore, the melanin production activity was severely affected by some of the point mutations. These are Leu180Ser, Leu180Ala, Arg115Ala and Phe204Leu. See FIG. 10. These data demonstrate that at least residues Leu180 and Arg115 in the Type II BUPO's are essential for melanin-producing activity.

### Example 7: Hydroxylation of Indole

Representative enzymes were tested for their ability to form the blue pigment indigo by catalyzing the hydroxylation of indole.

Reactions were performed using 5 mM indole as substrate in a K-phosphate buffer pH 8 at room temperature. Reactions were started by the addition of 2 mM hydrogen peroxide. The formation of indigo as a strong blue color was observed for enzymes NobraUPO (Seq. no. 17) and NotUPO (Seq. no. 16), and a light blue color was observed for ActUPO (seq. no. 18), see FIG. 6, rows C, D. These findings indicate that these enzymes can be used for conversion of indole and substituted indoles for production of indigoid dyes, using either whole cells, cell-free extract, or purified enzymes in a soluble or an immobilized form.

### Example 8: Oxidation of Primary Alcohols

Oxidation of veratryl alcohol (VA) to veratryl aldehyde is usually taken as a measure of lignin-peroxidase activity. It can be performed by measuring the absorbance of the formed aldehyde at 340 nm (c340=93 mM-lcm-1).

All enzymes (10 μL) were mixed with 170 μL of 5 mM VA in 50 mM KPi pH 8.0 and the reaction was started with addition of 20 μL 50 mM H₂O₂ (5 mM f.c.). Control reactions were set up without addition of H₂O₂. A change in absorbance was measured in the plate reader.

A clear confirmation of catalysis of VA oxidation was observed for Nobra (seq 17), Not (seq 16) and StrpetUPO (seq 15), Ppac (seq 12) and Act (seq 18). See FIG. 6, rows E, F.

### Example 9: Oxyfunctionalization of Indene, Thionaphthene and Sulfoxidation Reactions

All substrates were dissolved in 1 mL of methanol and diluted with 7 mL of KPi pH 7.5 to give 5 mM substrate solutions. Reaction setup consisted of mixing 400 μL of 50 mM KPi pH 7.5 containing 5 mM substrate, 50 μL of enzyme stock solution and 25-50 μL of 20 mM H₂O₂ (final concentration 1-2 mM). Reactions were incubated in Eppendorf tubes with orbital shaking (1-inch orbital, 150 rpm, 28° C.) for 1-24h, after which the reaction was stopped by extracting with ethyl-acetate (for GCMS or chiral GC analysis). Ethyl-acetate extract was dried over anhydrous sodium-sulphate and 1 μL was injected on HP5 column for GCMS or on Chiraldex G-TA column for chiral GC-FID analysis (both performed using Shimadzu instruments). Alternatively HPLC-UV/Vis with the chiral OD-H column can be used for quantification of enantiomeric excess.

Conversion of indene to 1,2-epoxyindane and 2-inden-2-one, as well as conversion of thionaphtene to hydroxy-benzothiophene and benzothiophen-2-one, was confirmed using GC-MS for enzymes with sequence numbers 16 and 17.

Sulfoxidation of various sulfide substrates (methyl phenyl sulfide, benzyl phenyl sulfide, allyl phenyl sulfide, benzyl methyl sulfide, N-butyl methyl sulfide, ethyl phenyl sulfide and isopropyl phenyl sulfide) was confirmed for enzymes with sequence numbers 4, 15, 16, 17 and 18 using GCMS analysis.

Moreover, using chiral GC and chiral HPLC analysis it was found that BUPOs are able to catalyze the enantioselective sulfoxidation of thioanisole (data not shown). This is another proof of true peroxygenase activity as it shows that oxygen is transferred from the heme iron. The enzymes tested were found to mainly produce the S-enantiomer (enzyme 18: 42% ee, enzyme 17: 23% ee, enzyme 16: 33% ee).

TABLE 1
Type II enzymes having peroxygenase/
pigment-producing activity

| | NCBI | | |
|---|---|---|---|
| Seq. | accession | | Amino acid |
| No. | number | source | sequence |
| 15 | NEA50176.1 | Streptomyces | MQPQLLFMPQVGHPY |
| | | sp. | QRPAEPPHTPGTAER |
| | | SID10815 | ELPEYDLLGARPVDA |
| | | | QRLFWYRWIAGHQIS |
| | | | FVLWRAMGDILWRHP |
| | | | DDPPGDRELDALATC |
| | | | VDGYSAMLLYSATVP |
| | | | RAHYHAHTRARMALQ |
| | | | HPSFSGAWAPDYRPV |
| | | | RRIFRGRFPWQGGRS |
| | | | CEALDQAIARNGVTH |
| | | | DHIANHLVPDGRSLL |
| | | | QQSAGAPGVSVSREK |
| | | | EDLYDNFFLTVRRPV |
| | | | GHTELVAQLDTRVAE |
| | | | VATDLAHNGLYPDVD |
| | | | GRHHPVVTWQSAGVM |
| | | | EPLLTGVLRVLDRAT |
| | | | RLMAHLRLEEVRS |
| 16 | WP_ | Nocardia | MQPHVLFMPPLGDSF |
| | 040743483.1 | tenerifensis | DGSAESDHSTEVTDR |
| | | | DWPRYNVFGNEPVEP |
| | | | ERLFWYRWIAGHQVS |
| | | | FLLWRAMCDVVWHHP |
| | | | DEDAPSERELDLLCA |
| | | | CIDGYSAVLLYSSTV |
| | | | PRDQYHADIRPRMAL |
| | | | QHPAFSGTWAPDYRP |
| | | | VRRLFRGKMPWQEDA |
| | | | SCAALGEAVARNGIT |
| | | | HSHIADHLVPEGRSL |
| | | | LQESAGAPGVSVSRE |
| | | | KEDLYDNFFLTVRRP |
| | | | VSQTEFVAQLDSRLA |
| | | | DLAADLAHNGLYPNV |
| | | | DGAHHPVVTEQADDE |
| | | | MRPFVTGVLDVLDRA |
| | | | ARLVSEMRLEEAHR |
| 17 | WP_ | Nocardia | MQPRLLFMPSLGEPF |
| | 029903812.1 | brasiliensis | DESAPDEDVVLAARD |
| | | | APEYPLFGTEPVEAE |
| | | | RLFWYRWIAGHQISF |
| | | | LLWRAMCDVVCQYPD |
| | | | EVPGERELEVLSACI |
| | | | DGYSAMLLYSSTVPR |
| | | | DHYHADIRPRMALQH |
| | | | PAFSGTWAPDYRPVR |
| | | | RLFHGRLPWQDDPSC |
| | | | RALDAAVDRNELTHS |
| | | | HIADHLVPDGRSLLQ |
| | | | QSVGALGVSVSREKE |
| | | | DLYDNFFLTVRRPVS |
| | | | HAEFVAQLDARITEL |
| | | | TVDLSHHGMYPMVDG |
| | | | RHHPVVGDRSEAVMQ |
| | | | PLITYAVQVVDRAAR |
| | | | LVAKMRLEGVRR |
| 18 | WP_ | Actinokineospora | MTHDRRRPQASTDPA |
| | 121392645.1 | cianjurensis | LQPRLLFLPATDGDY |
| | | | ADSDDTAHGADLDHL |
| | | | PDYTLFGTRPVEARR |
| | | | VFWYRWLAGHQISFA |
| | | | LWRAMSDVVTRRGDD |
| | | | LPSERELEVLTSCVD |
| | | | GYSAMLLYSATVPRD |
| | | | HYHANIRTRMALQHP |
| | | | SFSGAWAPDHKPIRL |
| | | | LFRGRFDWQDDPSCR |
| | | | ELDEAVARNRRTHDF |
| | | | IADHLVPDGRSLLQK |
| | | | SAGTVRQGVSRDKED |
| | | | LYDNFFLTIRRPVGH |
| | | | AELVTQLDDRVAELA |
| | | | EDLAHHGLYPEVDGR |
| | | | HHPVVTGRPDPALRP |
| | | | LIAEMPATLRRAAHL |
| | | | VAGMHYAGARS |

Type I enzymes having peroxygenase/pigment-producing activity

| | NCBI | | |
|---|---|---|---|
| Seq. | accession | | |
| No. | number | source | Amino acid sequence |
| 2 | WP_078759802.1 | Marinactinospora | MDWSPSQSADIPFHLGIPEELTALPPIGPVPDLVA |
| | | thermotolerans | PITERTGAIDVRGDLSLFAEAMRAARKTLPLPAPE |
| | | | PVEADPRESQANRDNDEAFGIVRVAGPPLLLMLDG |
| | | | LLKAAAGVVEAAADYGTRIPAEEWADLVHGFDTVL |
| | | | GWLGGDDRPSSPPPLAFPPEEGPESNADDGVLVRW |
| | | | VRGHHLFMAICQACALALAHARQAQQRGALASART |
| | | | ALGVAAVMMGSGRATLRFAADASPTDYTTRIRPTL |
| | | | MPPVAPPRMSGLHWRDHEELIRQIRAVSRSWAQFA |
| | | | RHAPGQVEEFESVLATTYDAHRNVCAHFVGEEPSL |
| | | | LASSGSRGPASLSGPSVLGRLRRQRLSLLSLPPRE |
| 3 | WP_033325725.1 | Streptomyces | MTDYLEHVATVPFRIARPADLPTTIPDLAVVGGAA |
| | | yerevanensis | AARVSELLLAPAARAGLPALCDAVRRAGDELGPVP |
| | | | STLVTDDPDESRPNRDNDAAFGIERHSGDPAQLLR |
| | | | LALLHALEEVLDLTAASGTGLDEETWRELVDGFDV |
| | | | LLHWLAHPDRVLPADAAARVPAAPAQRVSTPMEGL |
| | | | KRWVRGHHVFMPFSQGCGLALASMSDAAQRGDEDA |
| | | | AVVGARVAIRTMRAARAALCFAGDATATQYQDEIR |
| | | | PTLMPPVAPPQMSGLRWRDHEYLVERLTAAGPAWH |
| | | | WLAARGFEALLRDFLAALDGAYEAHKEVCAHFVGT |
| | | | ASPSLLATARSHRPAVGVIEQFRQIRRSAVSAPGE |
| | | | QPAAKEKP |
| 4 | AAL33759.1 | Pseudomonas | MESIAFPIAHKPFILGCPENLPATERALAPSAAMA |
| | | fluorescens | RQVLEYLEACPQAKNLEQYLGTLREVLAHLPCAST |
| | | | GLMTDDPRENQENRDNDFAFGIERHQGDTVTLMVK |
| | | | ATLDAAIQTGELVQRSGTSLDHSEWSDMMSVAQVI |
| | | | LQTIADPRVMPESRLTFQAPKSKVEEDDQDPLRRW |
| | | | VRGHLLFMVLCQGMSLCTNLLISAAHDKDLELACA |
| | | | QANRLIQLMNISRITLEFATDLNSQQYVSQIRPTL |
| | | | MPPIAPPKMSGINWRDHVVMIRWMRQSTDAWNFIE |
| | | | QAYPQLAERMRTTLAQVYSAHRGVCEKFVGEENTS |
| | | | LLAKENATNTAGQVLENLKKSRLKYLKTKGCAGAG |
| 5 | WP_157246732.1 | Nonomuraea sp. | MQDYLQGVVTEPFTLCRPDDLPGTTGELSSAAGLV |
| | | p1410 | RPGARTVTGPGARRDLAGLLAALGEARSALDRGPV |
| | | | TPVGEDPTESDLNKANDLGFGILRVRGEPVRVLTD |
| | | | AALRSIAAVLGLAARNGTGFEPATWRRLVGGFDAL |
| | | | LLWLADPGRAPASHPVPAPGERQPLEPGGALRRWI |
| | | | YGHHVFMVFSQGCTLSLACLRERAAAGDGSGAAAA |
| | | | AATAVRLMWASQGALSFAGDVAMEDYATEIRPTLM |
| | | | PPIAPPRMTGLHWRDHEALVRELAAARDAWQWLAG |
| | | | SRPELLEEFRDAIDSAYSAHRGVCASFVGDRSPSL |
| | | | LATSRSSRPAVAVLEQFHRRRLDMLPPSARDDGDG |
| | | | R |
| 6 | QCC21379.1 | Candidatus | MREISPIFYLLQPENLPISRDRLRCGDIVQEVLSH |
| | | Endohaliclona | ILNLTRNENTILDGIHQAFSLLAPSFEGISVVKED |
| | | renieramycinifaciens | PLESLENRANDEAFNIKRVNGNPSYVLTYSTLQSY |
| | | | AGMLEDALDIGTSLSEVEWTKLNLAYISIFDMLRG |
| | | | GITPINSFKFSTIMPKKSRTYNNENERDGMRRWVC |
| | | | GHYVFMSIVQAMIVALNFFSSEVANDDIQASQLAI |
| | | | DRAILLMKGSESALKFTGDFSRRSYEQSVRPSLSP |
| | | | PKAPRGMSGVNWRDHEYLIKKVFRKLQPIFLNPPL |
| | | | EVKAKLYDFLDALKDAYDAHKLVCSFFVGNDKPSL |
| | | | LSKKSAMNMLESYKQNRVAVLKKPQKGLRV |
| 7 | WP_152646293.1 | Streptacidiphilus | MIDVETQARHPDVPALHLLKAEQLPASMSAIIGMM |
| | | albus | NQRTADALRTVIDTNPQNSDRHALAAVDAALACLT |
| | | | APAATTKYTDAERNSLPEDLYEEHDLFFGAIRVTG |
| | | | NPVETFAHTMLQALRSQLNDALSAGTSLCSGERGQ |
| | | | LREAFWSVFHELWRIGVAGTPFEYAARARPLDTGL |
| | | | GLDDAEADPLIRWRLGHQVFFALIQALIVSVGCLE |
| | | | ECLRDDPDDIESACRLLEDATVLMIGSGASMRYAG |
| | | | DFTRTHYTDAVRPAMMPPHINAKFSGLQLRDHRIL |
| | | | LKLLNRVKPLLASPAPAVDKSYRQLLDAMSTAYDA |
| | | | HISVCSRFGGDRESSLRTPGSSLPAVGVLERFRSR |
| | | | RIGAATPDRPAE |
| 10 | WP_093605566.1 | Lentzea | MTLILLHPTSLPRREDQLSLAGDAPAPLRLARRLA |
| | | waywayandensis | AGVSGADLLDRLRTLPARSGAVALAERVDSGVPYT |
| | | | YDHRLREFDDHFGVERVDVDTDGVFARSLLVAYRS |
| | | | LLEEGLASGTRMSWARWSVLVSVLQNMIVRCAGEE |
| | | | ADLPVAVRPPLLRWHLDPERRWRVGHHVFFVVTQC |
| | | | LIVALQSFATALGEDDLPAARRALRLATRLLDASS |
| | | | AAFVFTAEFGANQYHQAVRPTMEPPFVSAGFSGLL |
| | | | SPDHHYLVRLFAEVRPMLRQLPADLVADHAAFVRA |
| | | | LGAVYESHKYVCARFGGDTGVSLRTSALPAVEVLH |
| | | | ALKLARTKIVGRP |
| 11 | WP_156139503.1 | Sphingomonas sp. | MVALEGLLARSDERTDTSSLDAPAAPPRRGSQIAR |
| | | 35-24ZXX | KGKVRKGGVKQVQAKLARVFLRGVEPVVVRSAPTS |
| | | | GSRLPRADLRGLTTALTANPKSHQHVVHRVRSRLV |
| | | | HIHALPATHVPTPSQVQAALGHNRLQQYAELMPER |
| | | | RTAHVLAALSTQLARPAQSLSAVVDSPNVRDNELH |
| | | | DAYFCVVRVDPRNLASHVEGIILAAQALRREMRSG |
| | | | SDRLGIADFGEIADGIELAFTLATGIRSPRIAMLR |
| | | | TVHRAALSETMIARRWMRGHQLFLVITQAMIASLN |
| | | | NLEVIAGHGSDDSEMAGAIEQLVRLLRAAAGSMRL |
| | | | TGDFPPDIYDTIIRPSMAPPELPDGFSGIFSSDHR |
| | | | FLVTRLREITPKFSGLKERLHDAHALLVKAFADLY |
| | | | DDHINVCANFVSSEKNSLLMADGARCTALEQLGKF |
| | | | KKSRLRLLGKP |
| 12 | WP_092059473.1 | Poseidonocella | MQVESLTLPLPHDWFAAETTLPKHRFRMSQLMASG |
| | | pacifica | NAMTGLAARVTQAVANEEFAPSESTLDGAQRLFDS |
| | | | YFLVERASGAPVEMLRAAMAKALPLCAADIETGSA |
| | | | LSDVQLQTLARGLHRLADWALIPTAAPAAASPHVT |
| | | | ADPLFRWKQQHQLFFLIIHGMLYLLHVLEETLDRE |
| | | | HAPVTQAVLSDFADLMEASTVAFHLAADFSPEDYE |
| | | | SRIRPDMTAHDPHFSGLFYADHKELVTSLRVLKRV |
| | | | PDDFEEELDRISRAISETYDAHARVCLRFVGETAS |
| | | | LASKDDSRIAAESIRGKYVKRTKVIAGLAKPRA |
| 13 | PKW18123.1 | Saccharopolyspora | MSCEKWNTMPDEQKADSFLRLLDPNHLPVERESLA |
| | | spinosa | RAAIRLVGDHRDDQVLRVTEPERHRARRVLTDELL |
| | | | GHEPIPPARSGRRALDQDEHDRHFGVVRVHGDSGA |
| | | | IFLTSLLVSYLDMLDDVERGGTTLGQAQWTDLLRV |
| | | | PTLVFDFALRRQASAFPGSDFPIPAGRCELLPHRR |
| | | | WHVGHRLFFTLIQTTTQAVTSLSQAVRAGDEAAEA |
| | | | DVRGCVDFVRLTTLGSTAALRLTADFSPVDYEQRV |
| | | | RPTMLPPHVRPGFSGLQTRDHRHLLNAMRKLREDM |
| | | | ATVAELGEPHAELIAAIRSLHDAHAYVCTRFGGDT |
| | | | APSLRMAALGQQDGKGVEVVRGLARHRESLLEPSR |
| | | | ANAHDQPRAYQRSDEDGHE |
| 19 | WP 149740153.1 | Rhizobium sp. RU20A | MPEDLPGRTAFCAAIPATLRADCAADIAASHMQFR |
| | | | RTLPALKALMPSAAAIETGRIDFMIDTGQACDIDS |
| | | | HDHFFRIRRSRTGAAPALLASQIQTLQILKTEVTA |
| | | | GPVTVPAERIVAMADAIRAAIAFLVGTQATTPTEL |
| | | | TFRSVTEGDPPAERAMQRWVRGHQIFAGLCQSLVV |
| | | | ALGALESAVQDQDDATVDSAGTLVASLLSASATAL |
| | | | ELTGDFPEARYNAGIRVAMDAPYFPTGFSGVLSRD |
| | | | HRQLVSRMKMMRPAIESLGRRHPALHASIGAALSS |
| | | | VYASHKHVCARFVEPRSTSLLMAATAERPATEQID |
| | | | RFRAMRLRAWDVSAEHPGVSGSSSE |
| 20 | WP_015586201.1 | Rhodococcus | MTPSPTSIDRVVLPTDSTIPVARSAEYDTYFRISR |
| | | | SNDPHAWAVSALTVVTAITTDLPQSTLDNDSRLRL |
| | | | LDALAVVVGDASAAAAVSARERDPSQLAAPTDHSA |
| | | | DNRTPQAPNDSARQAITRWKKGHHLFHLFVISMNS |
| | | | RLAAVLECLANKQWPTLAHQLTELGTLYAGATESM |
| | | | RYASDFSPEIYRDFVRPSMEPPWLEPGFSAVFNRD |
| | | | HDVMLTRAKIVRTTLKTAELPGVAADSARRLWKSQ |
| | | | AENRRAHQLICQRFVPGGDSLLQEHFDSTGQSN |
| 21 | WP_019808821.1 | Saccharomonospora | MNTENKTDLIETIALRDDARAPTSETAADSRHYDD |
| | | halophila | YFRIDRTDDSATWWAGAQRVARLVVDDLPATGLDE |
| | | | DLRADLLAGVSRLLLASASAPAHSGTTMPVRTPTH |
| | | | GEDSVAARRWKLGHHLFHHMLSLMNSHADGIASAL |
| | | | DEPDPVTVGLLVEELTVLYDSATATMHYASSFPIS |
| | | | AYAETVRPSMEPPWMPPGFSGVFNREHETLLSSLT |
| | | | GLKPRLRGRTAERSLPADVKTASRALWKAQSRNRR |
| | | | EHKMICERFVPGGGSLLQDYFDETRKG |
| 23 | WP_091674400.1 | Amycolatopsis marina | MTDADAPESARIAMRRWRLGHHAFHLQLVVMNTLL |
| | | | TRADQACAEGRWADLTSDLVRLRTLYNGATATMAL |
| | | | AAAFDSDMYNQVVRPSMEPPFLAPGFSAAFGREHT |
| | | | IMLRRIERLHHRIRAGSSTKEGRPDQVLRAHRELQ |
| | | | RAQARNRREHVLICRRFVQDGKSLLRESEATH |
| 24 | WP_098424542.1 | Bacillus | MIKQITEAHQQAMVRRKQGHQVFHVILVCMNTLLE |
| | | thuringiensis | ESKSALEQNNFLKLQSILEQLTVLYDSATVSMKYT |
| | | | ADFSPKYYEEMIRPSMMPPFLSPGFSGTQNKDHQQ |
| | | | MIEGLGQLQKQMLNKLGDKNKWPDNVLQAWNLLSK |
| | | | SQIRNRKHHGLVCQNHVPEGASLLKQFYQNNK |
| 25 | WP_162035492.1 | Chengkuizengella | MQSNSVESISTAYQQAVLRWKQGHQSFQIIIITMN |
| | | sediminis | TLLESSIQALDQNEWHLLTKSLDRLSNLMKASTAT |
| | | | MKYTSDFSPKSYEELIRPSMMPPFLSEGFSGVLNI |
| | | | DHKLMLIKFRKLRDLMVQKLGDKHQWPSMITRSWN |
| | | | HLMDAQTHNREHHGLVCQHFVDDGVSLMQNFYKEK |
| | | | REAKN |
| 26 | WP_156623984.1 | Mycobacterium sp. | MEQSTAALLSPEQKTAIRRWKLGHHIFHIYLFAMN |
| | | 852002- | TNLHTAREQLHRESWDQLVDSLQSLVQLYDAATAS |
| | | 40037_SCH5390672 | MKYAADMDRAEYQSLIRPSMAPPFLKPGFSGQQNK |
| | | | DHTTMVQLIRKLRREIRERGHRDNAYLPQEVYAAA |
| | | | KALWEAQARNRRHHILVCDRLVPGEGSLLQTYFAS |
| | | | IRSRGISKEGN |
| 27 | WP_093637903.1 | unclassified | MHISAEQQTAVRRWKLGHHVFHLHLTVMNTYLASL |
| | | Streptomyces | EKSINEEDWRSVSPLLTKLSRLYGAATSCMRYASD |
| | | | FPETAYESLIRPSMEPPWLNPGFSGKFNSDHERML |
| | | | DLMRTIRTSLKRAIRSGEVPEEVERAATQLWRAQS |
| | | | HNRANHKLICEKFVPGGQSLLQDYFNANA |
| 28 | WP_093231630.1 | Thermoflavimicrobium | MSVTNPSIPASYQQAVLRWKQGHHVFHVILVTMNT |
| | | dichotomicum | CLEESLRALNQQDWSRLIQLLERLATLYDAATATM |
| | | | KYSSNFSRKYYEEVIRPSMMPPFLKPGFSGKLNRE |
| | | | HNVMLDLFQTLRAELKKKEELPLGVEEAWRKLVQS |
| | | | QKRNRKHHGLVCQQFVDDGVSLLQEFYRSQTK |
| 29 | ADQ55481.1 | uncultured organism | MIIIQGLIISLQCFIFEYSKKKFLQSLLYLNNAIK |
| | | | LMKATEVALYYTGEFSSKSYNENVRPTLMPPISQP |
| | | | EMSGLNWRDHQFMVKNCMRSIGKLNFTSYPIIQKK |
| | | | YNIFIISLKKAYHAHKYVCGKFVGPASGSLRSNEY |
| | | | SAVQEIEKFKKLRLKILKG |

What is claimed is:

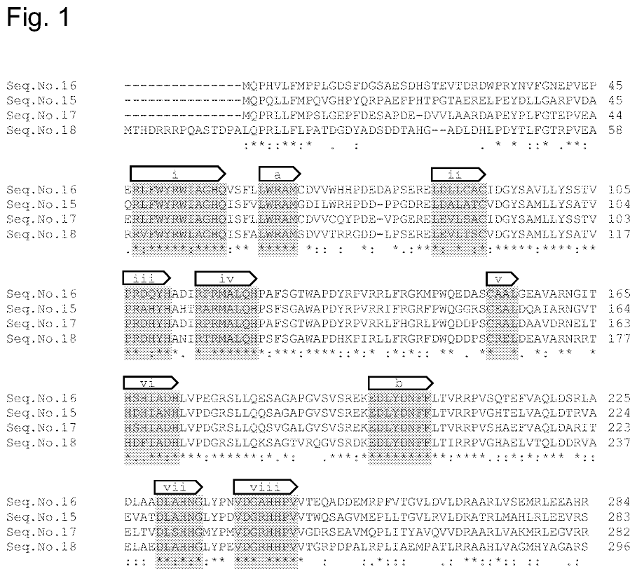

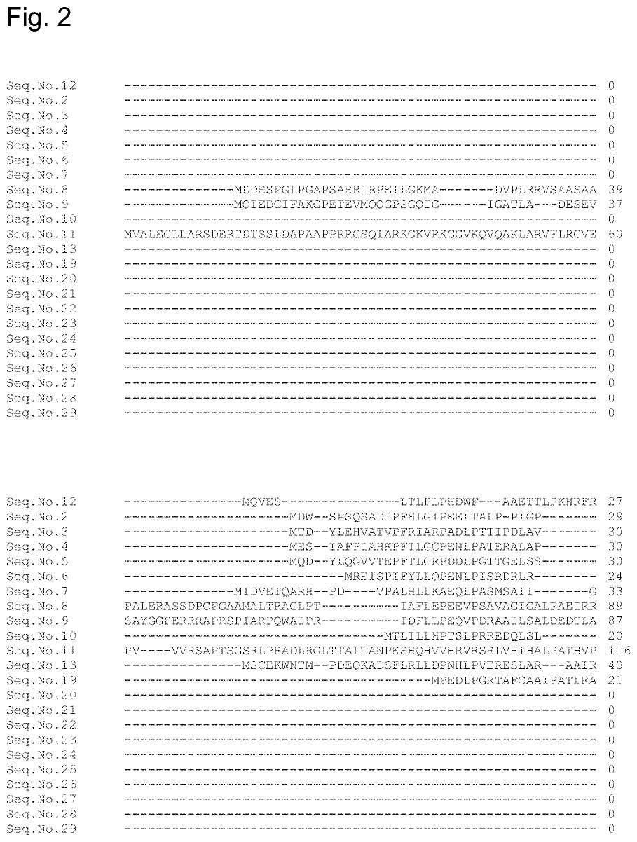

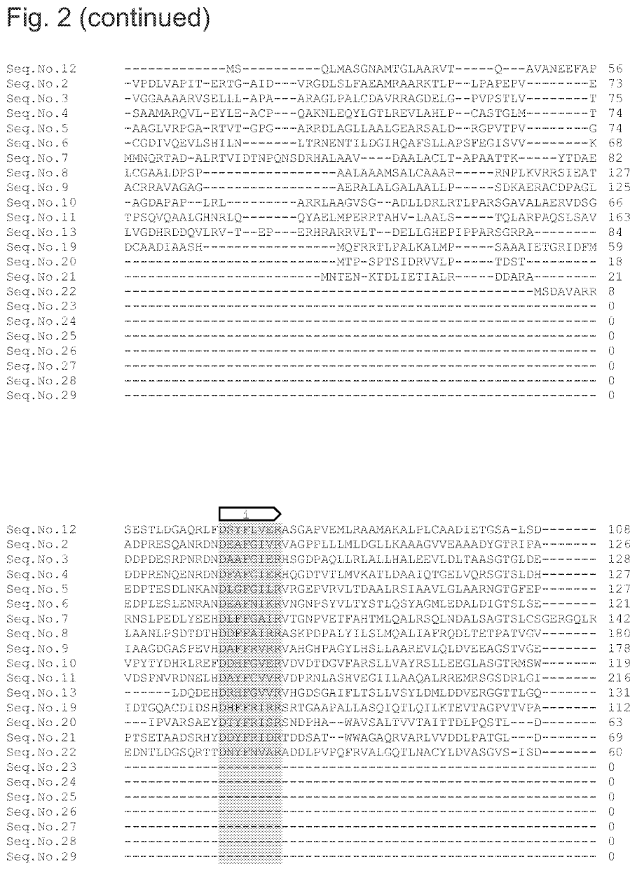

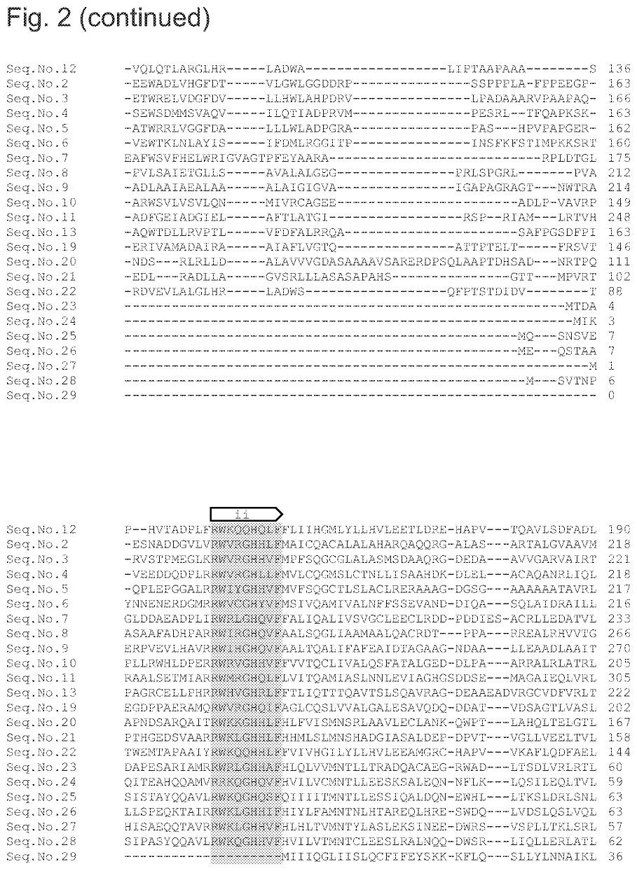

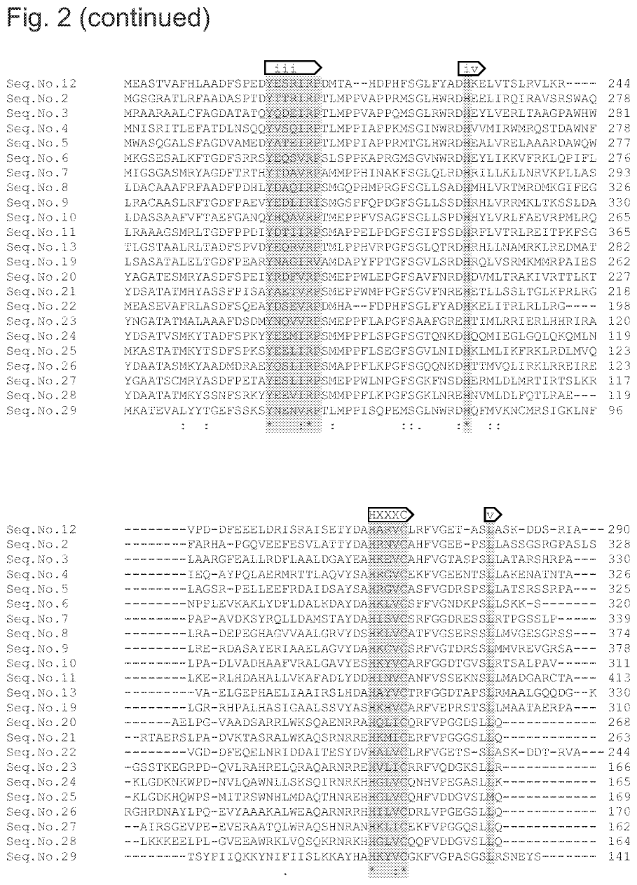

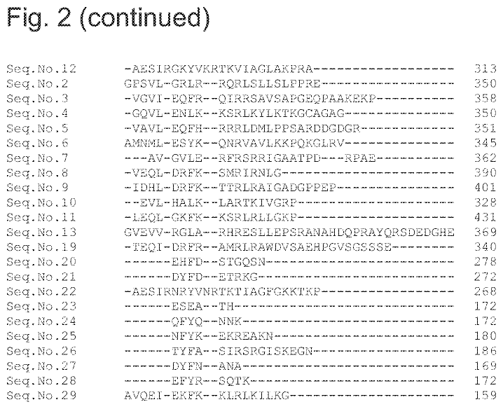

1. A method for the production of melanin and/or melanin-like pigment(s), comprising the use of a polypeptide selected from the group consisting of:
(a) a polypeptide comprising an amino acid sequence having at least 50% pairwise sequence identity when aligned to at least 200 consecutive amino acid residues of Seq. no. 16 of FIG. 1, and comprising at least two of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV;
wherein X is any amino acid;
(b) a polypeptide comprising an amino acid sequence having at least 30% pairwise sequence identity when aligned to at least 150 consecutive amino acid residues of Seq. no. 12 of FIG. 2, and comprising the motif HXXXC, wherein X is any amino acid, preferably H[IRKAQVG][GNELSYRHM][VI]C, more preferably HARVC;
(c) a fragment of the polypeptide of (a) or (b) that has pigment producing activity,
wherein the method comprises the fermentative production of melanin and/or a melanin-like pigment, the method comprising the steps of:
i) providing a microbial host cell expressing the heterologous polypeptide having piment producing activity;
ii) culturing the host cell in a culture medium and allowing for the production of melanin and/or melanin-like pigment; and
iii) isolating melanin and/or melanin-like pigment.

2. The method of claim 1, comprising the hydroxylation of L-tyrosine to L-DOPA and subsequent oxidation to dopachrome and the formation of melanin and/or a melanin-like pigment.

3. (canceled)

4. A method according to claim 3, wherein the host cell is a bacterial, yeast or fungal host cell, preferably a bacterial host cell, preferably E. coli.

5. The method according to claim 3, wherein the culture medium comprises at least one melanin(-like) pigment precursor, preferably L-Tyrosine, optionally in combination with L-cysteine.

6. A method for the hydroxylation or oxidation of a substituted or unsubstituted, linear or branched, aliphatic or aromatic substrate, comprising contacting the substrate with a polypeptide having peroxygenase activity and a source of hydrogen peroxide, wherein said polypeptide is selected from the group consisting of:
(a) a polypeptide comprising an amino acid sequence having at least 50% pairwise sequence identity when aligned to at least 200 consecutive amino acid residues of Seq. no. 16 of FIG. 1, and comprising at least two of the following motifs:
i) RXFWXRWXXGHQ, preferably R[LV]FWYRWIAGHQ;
ii) LXXLXXCXD, preferably L[DE][ALV]L[ACST][TAS]C[IV]D;
iii) PRXXYH, preferably PR[AD][HQ]YH;
iv) RXR[ML]ALQH, preferably R[APT]R[ML]ALQH;
v) CXXL, preferably C[EAR][AE]L;
vi) HXXIAXH, preferably H[DS][HF]IA[ND]H;
vii) DLXHXG, preferably DL[AS]H[NH]G; and
viii) VDGXHHPV, preferably VDG[AR]HHPV;
wherein X is any amino acid;
(b) a polypeptide comprising an amino acid sequence having at least 30% pairwise sequence identity when aligned to at least 150 consecutive amino acid residues of Seq. no. 12 of FIG. 2, and comprising the motif HXXXC, wherein X is any amino acid, preferably H[IRKAQVG][GNELSYRHM][VI]C, more preferably HARVC;
(c) a fragment of the polypeptide of (a) or (b) that has peroxygenase activity.

7. The method according to claim 6, comprising one or more of the following reactions:
the enantioselective sulfoxidation of an optionally substituted alkyl sulfide, aryl sulfide or aryl alkyl sulfide substrate, preferably the enantioselective sulfoxidation of a substrate selected from the group consisting of methyl phenyl sulfide, benzyl phenyl sulfide, allyl phenyl sulfide, benzyl methyl sulfide, N-butyl methyl sulfide, ethyl phenyl sulphide and isopropyl phenyl sulphide;
the manufacture of a substituted or unsubstituted indigo dye by contacting a substituted or unsubstituted indole substrate with a source of hydrogen peroxide and the polypeptide;
the oxidation of a primary alcohol, preferably the oxidation of veratryl alcohol to veratryl aldehyde.

8. (canceled)

9. (canceled)

10. (canceled)

11. (canceled)

12. The method according to claim 1, wherein the polypeptide is comprised in whole cells or a cell-free extract, or wherein the polypeptide is used as purified enzyme.

13. The method according to claim 1, wherein the polypeptide under (a) furthermore comprises one or both of the following motifs:
a. LWRAM
b. EDL[YF]DN[FY][FY], preferably EDLYDNFF.
and/or contains one or more of the residues corresponding to Arg115, Met118, Phe125, Ser126, Leu180, Tyr200 and Phe204 of Seq. no. 16.

14. (canceled)

15. The method according to claim 1, wherein the polypeptide comprises a sequence that has at least 60%, at least 70%, at least 80%, or at least 90% pairwise sequence identity with any one of Seq. no. 15, 16, 17 and 18 of Table 1, or a fragment thereof that has peroxygenase activity.

16. The method according to claim 1, wherein the polypeptide under (b) furthermore comprises one or more of the following residues/motifs:
i) DXXFXXXR; preferably D[LEAFDSRHT][AGFHY]F[GNCLR][IAV][VELKIRSD]R, more preferably DSYFLVER;
ii) R[WR]XX[GQ]HXXF; preferably R[WR][VIRMKH][RYCLVQK][GQ]-H[HLYQR][VLIAS]F, more preferably RWKQQHQLF;
iii) YXXXXR[PV]; preferably Y[N/T/Q/V/E/A/H/D/R/Q][E/T/D/S/Q/A]-[Q/R/E/S/A/I/G/F/T/V/M/L/N][IV]RP, more preferably YESRIRP;
iv) H232;
v) [LM]281; preferably L281
wherein X is any amino acid and wherein the numbering corresponds to the amino acid sequence of Seq. no. 12, and preferably, wherein in addition, comprises a sequence that has at least 40%, at least 50%, at least 60%, or at least 70% pairwise sequence identity with any one of Seq. no. 2-13 and 19-29 of Table 2, or a fragment thereof that has peroxygenase activity.

17. (canceled)

18. The method of claim 15, wherein the polypeptide is any one of Seq. no. 2, 3, 4, 7, 8, 11, 12, 13, 20, 21 and 28, or a fragment thereof that has peroxygenase activity.

19. The method according to claim 1, wherein the polypeptide is any of Seq. no. 2, 8, 11, 12, 13, 15, 16, 17, 18 and 20, preferably Seq. no. 15, 16 or 17, or a fragment thereof having pigment producing activity.

20. The method according to claim 1, wherein the polypeptide further comprises an N- and/or C-terminal protein tag allowing for enhanced expression, solubilization, purification and/or immobilization.

21. (canceled)

22. (canceled)

23. The method according to claim 6, wherein the polypeptide is comprised in whole cells or a cell-free extract, or wherein the polypeptide is used as purified enzyme.

24. The method according to claim 6, wherein the polypeptide under (a) furthermore comprises one or both of the following motifs:
a. LWRAM
b. EDL[YF]DN[FY][FY], preferably EDLYDNFF,
and/or contains one or more of the residues corresponding to Arg115, Met118, Phe125, Ser126, Leu180, Tyr200 and Phe204 of Seq. no. 16.

25. The method according to claim 6, wherein the polypeptide comprises a sequence that has at least 60%, at least 70%, at least 80%, or at least 90% pairwise sequence identity with any one of Seq. no. 15, 16, 17 and 18 of Table 1, or a fragment thereof that has peroxygenase activity.

26. The method according to claim 6, wherein the polypeptide under (b) furthermore comprises one or more of the following residues/motifs:
i) DXXFXXXR; preferably D[LEAFDSRHT][AGFHY]F[GNCLR][IAV][VELKIRSD]R, more preferably DSYFLVER;
ii) R[WR]XX[GQ]HXXF; preferably R[WR][VIRMKH][RYCLVQK][GQ]-H[HLYQR][VLIAS]F, more preferably RWKQQHQLF;
iii) YXXXXR[PV]; preferably Y[N/T/Q/V/E/A/H/D/R/Q][E/T/D/S/Q/A]-[Q/R/E/S/A/I/G/F/T/V/M/L/N][IV]RP, more preferably YESRIRP;
iv) H232;
v) [LM]281; preferably L281
wherein X is any amino acid and wherein the numbering corresponds to the amino acid sequence of Seq. no. 12, and preferably, wherein in addition, the polypeptide comprises a sequence that has at least 40%, at least 50%, at least 60%, or at least 70% pairwise sequence identity with any one of Seq. no. 2-13 and 19-29 of Table 2, or a fragment thereof that has peroxygenase activity.

27. The method of claim 25, wherein the polypeptide is any one of Seq. no. 2, 3, 4, 7, 8, 11, 12, 13, 20, 21 and 28, or a fragment thereof that has peroxygenase activity.

28. The method according to claim 6, wherein the polypeptide further comprises an N- and/or C-terminal protein tag allowing for enhanced expression, solubilization, purification and/or immobilization.


SEQUENCE LISTING (60 sequences)

SEQ ID NO: 1 - protein, 9 aa, Artificial Sequence
  placeholder sequence
AAAAAAAAA

SEQ ID NO: 2 - protein, 350 aa, Marinactinospora thermotolerans
MDWSPSQSADIPFHLGIPEELTALPPIGPVPDLVAPITERTGAIDVRGDLSLFAEAMRAARKTLPLPAPEPVEADPRESQANRDNDEAFGIVRVAGPPLLLMLDGLLKAAAGVVEAAADYGTRIPAEEWADLVHGFDTVLGWLGGDDRPSSPPPLAFPPEEGPESNADDGVLVRWVRGHHLFMAICQACALALAHARQAQQRGALASARTALGVAAVMMGSGRATLRFAADASPTDYTTRIRPTLMPPVAPPRMSGLHWRDHEELIRQIRAVSRSWAQFARHAPGQVEEFESVLATTYDAHRNVCAHFVGEEPSLLASSGSRGPASLSGPSVLGRLRRQRLSLLSLPPRE

SEQ ID NO: 3 - protein, 358 aa, Streptomyces yerevanensis
MTDYLEHVATVPFRIARPADLPTTIPDLAVVGGAAAARVSELLLAPAARAGLPALCDAVRRAGDELGPVPSTLVTDDPDESRPNRDNDAAFGIERHSGDPAQLLRLALLHALEEVLDLTAASGTGLDEETWRELVDGFDVLLHWLAHPDRVLPADAAARVPAAPAQRVSTPMEGLKRWVRGHHVFMPFSQGCGLALASMSDAAQRGDEDAAVVGARVAIRTMRAARAALCFAGDATATQYQDEIRPTLMPPVAPPQMSGLRWRDHEYLVERLTAAGPAWHWLAARGFEALLRDFLAALDGAYEAHKEVCAHFVGTASPSLLATARSHRPAVGVIEQFRQIRRSAVSAPGEQPAAKEKP

SEQ ID NO: 4 - protein, 350 aa, Pseudomonas fluorescens
MESIAFPIAHKPFILGCPENLPATERALAPSAAMARQVLEYLEACPQAKNLEQYLGTLREVLAHLPCASTGLMTDDPRENQENRDNDFAFGIERHQGDTVTLMVKATLDAAIQTGELVQRSGTSLDHSEWSDMMSVAQVILQTIADPRVMPESRLTFQAPKSKVEEDDQDPLRRWVRGHLLFMVLCQGMSLCTNLLISAAHDKDLELACAQANRLIQLMNISRITLEFATDLNSQQYVSQIRPTLMPPIAPPKMSGINWRDHVVMIRWMRQSTDAWNFIEQAYPQLAERMRTTLAQVYSAHRGVCEKFVGEENTSLLAKENATNTAGQVLENLKKSRLKYLKTKGCAGAG

SEQ ID NO: 5 - protein, 351 aa, Nonomuraea sp. p1410
MQDYLQGVVTEPFTLCRPDDLPGTTGELSSAAGLVRPGARTVTGPGARRDLAGLLAALGEARSALDRGPVTPVGEDPTESDLNKANDLGFGILRVRGEPVRVLTDAALRSIAAVLGLAARNGTGFEPATWRRLVGGFDALLLWLADPGRAPASHPVPAPGERQPLEPGGALRRWIYGHHVFMVFSQGCTLSLACLRERAAAGDGSGAAAAAATAVRLMWASQGALSFAGDVAMEDYATEIRPTLMPPIAPPRMTGLHWRDHEALVRELAAARDAWQWLAGSRPELLEEFRDAIDSAYSAHRGVCASFVGDRSPSLLATSRSSRPAVAVLEQFHRRRLDMLPPSARDDGDGR

SEQ ID NO: 6 - protein, 345 aa, Candidatus Endohaliclona renieramycinifaciens
MREISPIFYLLQPENLPISRDRLRCGDIVQEVLSHILNLTRNENTILDGIHQAFSLLAPSFEGISVVKEDPLESLENRANDEAFNIKRVNGNPSYVLTYSTLQSYAGMLEDALDIGTSLSEVEWTKLNLAYISIFDMLRGGITPINSFKFSTIMPKKSRTYNNENERDGMRRWVCGHYVFMSIVQAMIVALNFFSSEVANDDIQASQLAIDRAILLMKGSESALKFTGDFSRRSYEQSVRPSLSPPKAPRGMSGVNWRDHEYLIKKVFRKLQPIFLNPPLEVKAKLYDFLDALKDAYDAHKLVCSFFVGNDKPSLLSKKSAMNMLESYKQNRVAVLKKPQKGLRV

SEQ ID NO: 7 - protein, 362 aa, Streptacidiphilus albus
MIDVETQARHPDVPALHLLKAEQLPASMSAIIGMMNQRTADALRTVIDTNPQNSDRHALAAVDAALACLTAPAATTKYTDAERNSLPEDLYEEHDLFFGAIRVTGNPVETFAHTMLQALRSQLNDALSAGTSLCSGERGQLREAFWSVFHELWRIGVAGTPFEYAARARPLDTGLGLDDAEADPLIRWRLGHQVFFALIQALIVSVGCLEECLRDDPDDIESACRLLEDATVLMIGSGASMRYAGDFTRTHYTDAVRPAMMPPHINAKFSGLQLRDHRILLKLLNRVKPLLASPAPAVDKSYRQLLDAMSTAYDAHISVCSRFGGDRESSLRTPGSSLPAVGVLERFRSRRIGAATPDRPAE

SEQ ID NO: 8 - protein, 390 aa, Artificial Sequence
  Type I enzyme having peroxygenase activity
MDDRSPGLPGAPSARRIRPEILGKMADVPLRRVSAASAAPALERASSDPCPGAAMALTRAGLPTIAFLEPEEVPSAVAGIGLCGAALDPSPAALAAAMSALCAAARRNPLKVRRSIEATALPAEIRRLAANLPSDTDTHDDFFAIRRASKPDPALYILSLMQALIAFRQDLTETPATVGVPVLSAIETGLLSAVALALGEGPRLSPGRLPVAASAAFADHPARRWIRGHQVFAALSQGLIAAMAALQACRDTPPARREALRHVVTGLDACAAAFRFAADFPDHLYDAQIRPSMGQPHMPRGFSGLLSADHMHLVRTMRDMKGIFEGLRADEPEGHAGVVAALGRVYDSHKLVCATFVGSERSSLLMVGESGRSSVEQLDRFKSMRIRNLG

SEQ ID NO: 9 - protein, 401 aa, Artificial Sequence
  Type I enzyme having peroxygenase activity
MQIEDGIFAKGPETEVMQQGPSGQIGIGATLADESEVSAYGGPERRRAPRSPIARPQWAIPRIDFLLPEQVPDRAAILSALDEDTLAACRRAVAGAGAERALALGALAALLPSDKAERACDPAGLIAAGDGASPEVHDAFFRVRRVAHGHPAGYLHSLLAAREVLQLDVEEAGSTVGEADLAAIAEALAAALAIGIGVAIGAPAGRAGTNWTRAERPVEVLHAVRRWIHGHQVFAALTQALIFAFEAIDTAGAAGNDAALLEAADLAAITLRACAASLRFTGDFPAEVYEDLIRISMGSPFQPDGFSGLLSSDHRHLVRRMKLTKSSLDALRERDASAYERIAAELAGVYDAHKCVCSRFVGTDRSSLMMVREVGRSAIDHLDRFKTTRLRAIGADGPPEP

SEQ ID NO: 10 - protein, 328 aa, Lentzea waywayandensis
MTLILLHPTSLPRREDQLSLAGDAPAPLRLARRLAAGVSGADLLDRLRTLPARSGAVALAERVDSGVPYTYDHRLREFDDHFGVERVDVDTDGVFARSLLVAYRSLLEEGLASGTRMSWARWSVLVSVLQNMIVRCAGEEADLPVAVRPPLLRWHLDPERRWRVGHHVFFVVTQCLIVALQSFATALGEDDLPAARRALRLATRLLDASSAAFVFTAEFGANQYHQAVRPTMEPPFVSAGFSGLLSPDHHYLVRLFAEVRPMLRQLPADLVADHAAFVRALGAVYESHKYVCARFGGDTGVSLRTSALPAVEVLHALKLARTKIVGRP

SEQ ID NO: 11 - protein, 431 aa, Sphingomonas sp. 35-24ZXX
MVALEGLLARSDERTDTSSLDAPAAPPRRGSQIARKGKVRKGGVKQVQAKLARVFLRGVEPVVVRSAPTSGSRLPRADLRGLTTALTANPKSHQHVVHRVRSRLVHIHALPATHVPTPSQVQAALGHNRLQQYAELMPERRTAHVLAALSTQLARPAQSLSAVVDSPNVRDNELHDAYFCVVRVDPRNLASHVEGIILAAQALRREMRSGSDRLGIADFGEIADGIELAFTLATGIRSPRIAMLRTVHRAALSETMIARRWMRGHQLFLVITQAMIASLNNLEVIAGHGSDDSEMAGAIEQLVRLLRAAAGSMRLTGDFPPDIYDTIIRPSMAPPELPDGFSGIFSSDHRFLVTRLREITPKFSGLKERLHDAHALLVKAFADLYDDHINVCANFVSSEKNSLLMADGARCTALEQLGKFKKSRLRLLGKP

SEQ ID NO: 12 - protein, 313 aa, Poseidonocella pacifica
MQVESLTLPLPHDWFAAETTLPKHRFRMSQLMASGNAMTGLAARVTQAVANEEFAPSESTLDGAQRLFDSYFLVERASGAPVEMLRAAMAKALPLCAADIETGSALSDVQLQTLARGLHRLADWALIPTAAPAAASPHVTADPLFRWKQQHQLFFLIIHGMLYLLHVLEETLDREHAPVTQAVLSDFADLMEASTVAFHLAADFSPEDYESRIRPDMTAHDPHFSGLFYADHKELVTSLRVLKRVPDDFEEELDRISRAISETYDAHARVCLRFVGETASLASKDDSRIAAESIRGKYVKRTKVIAGLAKPRA

SEQ ID NO: 13 - protein, 369 aa, Saccharopolyspora spinosa
MSCEKWNTMPDEQKADSFLRLLDPNHLPVERESLARAAIRLVGDHRDDQVLRVTEPERHRARRVLTDELLGHEPIPPARSGRRALDQDEHDRHFGVVRVHGDSGAIFLTSLLVSYLDMLDDVERGGTTLGQAQWTDLLRVPTLVFDFALRRQASAFPGSDFPIPAGRCELLPHRRWHVGHRLFFTLIQTTTQAVTSLSQAVRAGDEAAEADVRGCVDFVRLTTLGSTAALRLTADFSPVDYEQRVRPTMLPPHVRPGFSGLQTRDHRHLLNAMRKLREDMATVAELGEPHAELIAAIRSLHDAHAYVCTRFGGDTAPSLRMAALGQQDGKGVEVVRGLARHRESLLEPSRANAHDQPRAYQRSDEDGHE

SEQ ID NO: 14 - protein, 9 aa, Artificial Sequence
  placeholder sequence
AAAAAAAAA

SEQ ID NO: 15 - protein, 283 aa, Streptomyces sp. SID10815
MQPQLLFMPQVGHPYQRPAEPPHTPGTAERELPEYDLLGARPVDAQRLFWYRWIAGHQISFVLWRAMGDILWRHPDDPPGDRELDALATCVDGYSAMLLYSATVPRAHYHAHTRARMALQHPSFSGAWAPDYRPVRRIFRGRFPWQGGRSCEALDQAIARNGVTHDHIANHLVPDGRSLLQQSAGAPGVSVSREKEDLYDNFFLTVRRPVGHTELVAQLDTRVAEVATDLAHNGLYPDVDGRHHPVVTWQSAGVMEPLLTGVLRVLDRATRLMAHLRLEEVRS

SEQ ID NO: 16 - protein, 284 aa, Nocardia tenerifensis
MQPHVLFMPPLGDSFDGSAESDHSTEVTDRDWPRYNVFGNEPVEPERLFWYRWIAGHQVSFLLWRAMCDVVWHHPDEDAPSERELDLLCACIDGYSAVLLYSSTVPRDQYHADIRPRMALQHPAFSGTWAPDYRPVRRLFRGKMPWQEDASCAALGEAVARNGITHSHIADHLVPEGRSLLQESAGAPGVSVSREKEDLYDNFFLTVRRPVSQTEFVAQLDSRLADLAADLAHNGLYPNVDGAHHPVVTEQADDEMRPFVTGVLDVLDRAARLVSEMRLEEAHR

SEQ ID NO: 17 - protein, 282 aa, Nocardia brasiliensis
MQPRLLFMPSLGEPFDESAPDEDVVLAARDAPEYPLFGTEPVEAERLFWYRWIAGHQISFLLWRAMCDVVCQYPDEVPGERELEVLSACIDGYSAMLLYSSTVPRDHYHADIRPRMALQHPAFSGTWAPDYRPVRRLFHGRLPWQDDPSCRALDAAVDRNELTHSHIADHLVPDGRSLLQQSVGALGVSVSREKEDLYDNFFLTVRRPVSHAEFVAQLDARITELTVDLSHHGMYPMVDGRHHPVVGDRSEAVMQPLITYAVQVVDRAARLVAKMRLEGVRR

SEQ ID NO: 18 - protein, 296 aa, Actinokineospora cianjurensis
MTHDRRRPQASTDPALQPRLLFLPATDGDYADSDDTAHGADLDHLPDYTLFGTRPVEARRVFWYRWLAGHQISFALWRAMSDVVTRRGDDLPSERELEVLTSCVDGYSAMLLYSATVPRDHYHANIRTRMALQHPSFSGAWAPDHKPIRLLFRGRFDWQDDPSCRELDEAVARNRRTHDFIADHLVPDGRSLLQKSAGTVRQGVSRDKEDLYDNFFLTIRRPVGHAELVTQLDDRVAELAEDLAHHGLYPEVDGRHHPVVTGRPDPALRPLIAEMPATLRRAAHLVAGMHYAGARS

SEQ ID NO: 19 - protein, 340 aa, Rhizobium sp. RU20A
MPEDLPGRTAFCAAIPATLRADCAADIAASHMQFRRTLPALKALMPSAAAIETGRIDFMIDTGQACDIDSHDHFFRIRRSRTGAAPALLASQIQTLQILKTEVTAGPVTVPAERIVAMADAIRAAIAFLVGTQATTPTELTFRSVTEGDPPAERAMQRWVRGHQIFAGLCQSLVVALGALESAVQDQDDATVDSAGTLVASLLSASATALELTGDFPEARYNAGIRVAMDAPYFPTGFSGVLSRDHRQLVSRMKMMRPAIESLGRRHPALHASIGAALSSVYASHKHVCARFVEPRSTSLLMAATAERPATEQIDRFRAMRLRAWDVSAEHPGVSGSSSE

SEQ ID NO: 20 - protein, 278 aa, Rhodococcus
MTPSPTSIDRVVLPTDSTIPVARSAEYDTYFRISRSNDPHAWAVSALTVVTAITTDLPQSTLDNDSRLRLLDALAVVVGDASAAAAVSARERDPSQLAAPTDHSADNRTPQAPNDSARQAITRWKKGHHLFHLFVISMNSRLAAVLECLANKQWPTLAHQLTELGTLYAGATESMRYASDFSPEIYRDFVRPSMEPPWLEPGFSAVFNRDHDVMLTRAKIVRTTLKTAELPGVAADSARRLWKSQAENRRAHQLICQRFVPGGDSLLQEHFDSTGQSN

SEQ ID NO: 21 - protein, 272 aa, Saccharomonospora halophila
MNTENKTDLIETIALRDDARAPTSETAADSRHYDDYFRIDRTDDSATWWAGAQRVARLVVDDLPATGLDEDLRADLLAGVSRLLLASASAPAHSGTTMPVRTPTHGEDSVAARRWKLGHHLFHHMLSLMNSHADGIASALDEPDPVTVGLLVEELTVLYDSATATMHYASSFPISAYAETVRPSMEPPWMPPGFSGVFNREHETLLSSLTGLKPRLRGRTAERSLPADVKTASRALWKAQSRNRREHKMICERFVPGGGSLLQDYFDETRKG

SEQ ID NO: 22 - protein, 268 aa, Artificial Sequence
  Type I enzyme having peroxygenase activity
MSDAVARREDNTLDGSQRTTDNYFNVARADDLPVPQFRVALGQTLNACYLDVASGVSISDRDVEVLALGLHRLADWSQFPTSTDIDVTTWEMTAPAAIYRWKQQHHLFFVIVHGILYLLHVLEEAMGRCHAPVVKAFLQDFAELMEASEVAFRLASDFSQEAYDSEVRPDMHAFDPHFSGLFYADHKELITRLRLLRGVGDDFEQELNRIDDAITESYDVHALVCLRFVGETSSLASKDDTRVAAESIRNRYVNRTKTIAGFGKKTKP

SEQ ID NO: 23 - protein, 172 aa, Amycolatopsis marina
MTDADAPESARIAMRRWRLGHHAFHLQLVVMNTLLTRADQACAEGRWADLTSDLVRLRTLYNGATATMALAAAFDSDMYNQVVRPSMEPPFLAPGFSAAFGREHTIMLRRIERLHHRIRAGSSTKEGRPDQVLRAHRELQRAQARNRREHVLICRRFVQDGKSLLRESEATH

SEQ ID NO: 24 - protein, 172 aa, Bacillus thuringiensis
MIKQITEAHQQAMVRRKQGHQVFHVILVCMNTLLEESKSALEQNNFLKLQSILEQLTVLYDSATVSMKYTADFSPKYYEEMIRPSMMPPFLSPGFSGTQNKDHQQMIEGLGQLQKQMLNKLGDKNKWPDNVLQAWNLLSKSQIRNRKHHGLVCQNHVPEGASLLKQFYQNNK

SEQ ID NO: 25 - protein, 180 aa, Chengkuizengella sediminis
MQSNSVESISTAYQQAVLRWKQGHQSFQIIIITMNTLLESSIQALDQNEWHLLTKSLDRLSNLMKASTATMKYTSDFSPKSYEELIRPSMMPPFLSEGFSGVLNIDHKLMLIKFRKLRDLMVQKLGDKHQWPSMITRSWNHLMDAQTHNREHHGLVCQHFVDDGVSLMQNFYKEKREAKN

SEQ ID NO: 26 - protein, 186 aa, Mycobacterium sp. 852002-40037_SCH5390672
MEQSTAALLSPEQKTAIRRWKLGHHIFHIYLFAMNTNLHTAREQLHRESWDQLVDSLQSLVQLYDAATASMKYAADMDRAEYQSLIRPSMAPPFLKPGFSGQQNKDHTTMVQLIRKLRREIRERGHRDNAYLPQEVYAAAKALWEAQARNRRHHILVCDRLVPGEGSLLQTYFASIRSRGISKEGN

SEQ ID NO: 27 - protein, 169 aa, unclassified Streptomyces
MHISAEQQTAVRRWKLGHHVFHLHLTVMNTYLASLEKSINEEDWRSVSPLLTKLSRLYGAATSCMRYASDFPETAYESLIRPSMEPPWLNPGFSGKFNSDHERMLDLMRTIRTSLKRAIRSGEVPEEVERAATQLWRAQSHNRANHKLICEKFVPGGQSLLQDYFNANA

SEQ ID NO: 28 - protein, 172 aa, Thermoflavimicrobium dichotomicum
MSVTNPSIPASYQQAVLRWKQGHHVFHVILVTMNTCLEESLRALNQQDWSRLIQLLERLATLYDAATATMKYSSNFSRKYYEEVIRPSMMPPFLKPGFSGKLNREHNVMLDLFQTLRAELKKKEELPLGVEEAWRKLVQSQKRNRKHHGLVCQQFVDDGVSLLQEFYRSQTK

SEQ ID NO: 29 - protein, 159 aa, uncultured organism
MIIIQGLIISLQCFIFEYSKKKFLQSLLYLNNAIKLMKATEVALYYTGEFSSKSYNENVRPTLMPPISQPEMSGLNWRDHQFMVKNCMRSIGKLNFTSYPIIQKKYNIFIISLKKAYHAHKYVCGKFVGPASGSLRSNEYSAVQEIEKFKKLRLKILKG

SEQ ID NO: 30 - protein, 12 aa, Artificial Sequence
  motif
  MISC_FEATURE (2)..(2): X is any amino acid
  MISC_FEATURE (5)..(5): X is any amino acid
  MISC_FEATURE (8)..(9): X is any amino acid
RXFWXRWXXGHQ

SEQ ID NO: 31 - protein, 12 aa, Artificial Sequence
  motif
  MISC_FEATURE (2)..(2): X is L or V
RXFWYRWIAGHQ

SEQ ID NO: 32 - protein, 9 aa, Artificial Sequence
  motif
  MISC_FEATURE (2)..(3): X is any amino acid
  MISC_FEATURE (5)..(6): X is any amino acid
  MISC_FEATURE (8)..(8): X is any amino acid
LXXLXXCXD

SEQ ID NO: 33 - protein, 9 aa, Artificial Sequence
  motif
  MISC_FEATURE (2)..(2): X is D or E
  MISC_FEATURE (3)..(3): X is A or L or V
  MISC_FEATURE (5)..(5): X is A or C or S or T
  MISC_FEATURE (6)..(6): X is T or A or S
  MISC_FEATURE (8)..(8): X is I or V
LXXLXXCXD

SEQ ID NO: 34 - protein, 6 aa, Artificial Sequence
  motif
  MISC_FEATURE (3)..(4): X is any amino acid
PRXXYH

SEQ ID NO: 35 - protein, 6 aa, Artificial Sequence
  motif
  MISC_FEATURE (3)..(3): X is A or D
  MISC_FEATURE (4)..(4): X is H or Q
PRXXYH

SEQ ID NO: 36 - protein, 8 aa, Artificial Sequence
  motif
  MISC_FEATURE (2)..(2): X is any amino acid
  MISC_FEATURE (4)..(4): X is M or L
RXRXALQH

SEQ ID NO: 37 - protein, 8 aa, Artificial Sequence
  motif
  MISC_FEATURE (2)..(2): X is A or P or T
  MISC_FEATURE (4)..(4): X is M or L
RXRXALQH

SEQ ID NO: 38 - protein, 4 aa, Artificial Sequence
  motif
  MISC_FEATURE (2)..(3): X is any amino acid
CXXL

SEQ ID NO: 39 - protein, 4 aa, Artificial Sequence
  motif
  MISC_FEATURE (2)..(2): X is E or A or R
  MISC_FEATURE (3)..(3): X is A or E
CXXL

SEQ ID NO: 40 - protein, 7 aa, Artificial Sequence
  motif
  MISC_FEATURE (2)..(3): X is any amino acid
  MISC_FEATURE (6)..(6): X is any amino acid
HXXIAXH

SEQ ID NO: 41 - protein, 7 aa, Artificial Sequence
  motif
  MISC_FEATURE (2)..(2): X is D or S
  MISC_FEATURE (3)..(3): X is H or F
  MISC_FEATURE (3)..(6): X is N or D
HXXIAXH

SEQ ID NO: 42 - protein, 6 aa, Artificial Sequence
  motif
  MISC_FEATURE (3)..(3): X is any amino acid
  MISC_FEATURE (5)..(5): X is any amino acid
DLXHXG

SEQ ID NO: 43 - protein, 6 aa, Artificial Sequence
  motif
  MISC_FEATURE (3)..(3): X is A or S
  MISC_FEATURE (5)..(5): X is N or H
DLXHXG

SEQ ID NO: 44 - protein, 8 aa, Artificial Sequence
  motif
  MISC_FEATURE (4)..(4): X is any amino acid
VDGXHHPV

SEQ ID NO: 45 - protein, 8 aa, Artificial Sequence
  motif
  MISC_FEATURE (4)..(4): X is A or R
VDGXHHPV

SEQ ID NO: 46 - protein, 5 aa, Artificial Sequence
  motif
  MISC_FEATURE (2)..(4): X is any amino acid
HXXXC

SEQ ID NO: 47 - protein, 5 aa, Artificial Sequence
  motif
  MISC_FEATURE (2)..(2): X is I or R or K or A or Q or V or G
  MISC_FEATURE (3)..(3): X is G or N or E or L or S or Y or R or H or M
  MISC_FEATURE (4)..(4): X is V or I
HXXXC

SEQ ID NO: 48 - protein, 5 aa, Artificial Sequence
  motif
HARVC

SEQ ID NO: 49 - protein, 8 aa, Artificial Sequence
  motif
  MISC_FEATURE (2)..(3): X is any amino acid
  MISC_FEATURE (5)..(7): X is any amino acid
DXXFXXXR

SEQ ID NO: 50 - protein, 8 aa, Artificial Sequence
  motif
  MISC_FEATURE (2)..(2): X is L or E or A or F or D or S or R or H or T
  MISC_FEATURE (3)..(3): X is A or G or F or H or Y
  MISC_FEATURE (5)..(5): X is G or N or C or L or R
  MISC_FEATURE (6)..(6): X is I or A or V
  MISC_FEATURE (7)..(7): X is V or E or L or K or I or R or S or D
DXXFXXXR

SEQ ID NO: 51 - protein, 8 aa, Artificial Sequence
  motif
DSYFLVER

SEQ ID NO: 52 - protein, 9 aa, Artificial Sequence
  motif
  MISC_FEATURE (2)..(2): X is W or R
  MISC_FEATURE (3)..(4): X is any amino acid
  MISC_FEATURE (5)..(5): X is G or Q
  MISC_FEATURE (7)..(8): X is any amino acid
RXXXXHXXF

SEQ ID NO: 53 - protein, 9 aa, Artificial Sequence
  motif
  MISC_FEATURE (2)..(2): X is W or R
  MISC_FEATURE (3)..(3): X is V or I or R or M or K or H
  MISC_FEATURE (4)..(4): X is R or Y or C or L or V or Q or K
  MISC_FEATURE (5)..(5): X is G or Q
  MISC_FEATURE (7)..(7): X is H or L or Y or Q or R
  MISC_FEATURE (8)..(8): X is V or L or I or A or S
RXXXXHXXF

SEQ ID NO: 54 - protein, 9 aa, Artificial Sequence
  motif
RWKQQHQLF

SEQ ID NO: 55 - protein, 7 aa, Artificial Sequence
  motif
  MISC_FEATURE (2)..(5): X is any amino acid
  MISC_FEATURE (7)..(7): X is P or V
YXXXXRX

SEQ ID NO: 56 - protein, 7 aa, Artificial Sequence
  motif
  MISC_FEATURE (2)..(2): Xis N or T or Q or V or E or A or H or D or R or Q
  MISC_FEATURE (3)..(3): X is E or T or D or S or Q or A
  MISC_FEATURE (4)..(4): X is Q or R or E or S or A or I or G or F or T or V or M or L or  N
  MISC_FEATURE (5)..(5): X is I or V
YXXXXRP

SEQ ID NO: 57 - protein, 7 aa, Artificial Sequence
  motif
YESRIRP

SEQ ID NO: 58 - protein, 5 aa, Artificial Sequence
  motif
LWRAM

SEQ ID NO: 59 - protein, 8 aa, Artificial Sequence
  motif
  MISC_FEATURE (4)..(4): X is Y or F
  MISC_FEATURE (7)..(8): X is F or Y
EDLXDNXX

SEQ ID NO: 60 - protein, 8 aa, Artificial Sequence
  motif
EDLYDNFF